(19) United States
(12) Patent Application Publication
(10) Pub. No.: US 2025/0177565 A1
(43) Pub. Date: Jun. 5, 2025

(54) METHODS AND MATERIALS FOR INHIBITING/PREVENTING THE TRAFFICKING OF MONOCYTES TO THE CENTRAL NERVOUS SYSTEM

(71) Applicant: THE TRUSTEES OF DARTMOUTH COLLEGE, Hanover, NH (US)

(72) Inventors: Matthew C. HAVRDA, Etna, NH (US); Karl BIGGS, West Lebanon, NH (US); Kathleen PAUL, West Lebanon, NH (US)

(21) Appl. No.: 18/828,030

(22) Filed: Sep. 9, 2024

Related U.S. Application Data
(63) Continuation of application No. PCT/US2023/064087, filed on Mar. 10, 2023.
(60) Provisional application No. 63/318,612, filed on Mar. 10, 2022.

(51) Int. Cl. A61K 48/00; A61K 45/06; A61P 25/16; A61P 29/00; A61P 37/06; C12N 9/22; C12N 15/11; C12Q 1/6883; G01N 33/569

(52) U.S. Cl. CPC A61K 48/005; A61K 45/06; A61P 25/16; A61P 29/00; A61P 37/06; C12N 9/22; C12N 15/111; C12Q 1/6883; G01N 33/56972; C12N 2310/20; C12Q 2600/158

(57) ABSTRACT

The present invention relates to preventing or inhibiting the formation of M2 monocytes and/or preventing or inhibiting monocyte trafficking/infiltration/migration to inflamed sites in a subject in need thereof, e.g., a subject having or at risk of developing an acute or chronic neuroinflammatory disease, by inhibiting the expression and/or activity of “BRI3” or “brain protein I3”.

### RELATED APPLICATIONS

This application claims priority as a continuation application to PCT Appl. No. PCT/US2023/064087 filed Mar. 10, 2023, which claims priority to U.S. Provisional Appl. No. 63/318,612 filed on Mar. 10, 2022, the contents of each and all of which are incorporated by reference in their entirety.

This invention was made with government support under Grants R01 ES024745-07 and R01 ES033462-02 awarded by the National Institutes of Health and National Institute of Environmental Health Sciences. The government has certain rights in the invention.

### SEQUENCE LISTING

This application includes as part of its disclosure an electronic sequence listing text file named “1143252o005003.xml”, having a size of 110,944 bytes and created on Sep. 6, 2024, which is hereby incorporated by reference in its entirety.

### FIELD OF THE INVENTION

The present invention relates to preventing or inhibiting the formation of M2 monocytes and/or preventing or inhibiting monocyte trafficking/infiltration/migration to inflamed sites in a subject in need thereof, e.g., a subject having or at risk of developing an acute or chronic neuroinflammatory disease, by inhibiting the expression and/or activity of “BRI3” or “brain protein 13”.

### BACKGROUND OF THE INVENTION

Monocytes are heterogeneous circulating blood cells which under certain circumstances rapidly extravasate into inflamed tissues including in the central nervous system. In the bone marrow (BM), a common Lin⁻ckithiCD115⁺CX3CR1⁺Flt3⁺ progenitor cell, termed macrophage and DC precursor (MDP), gives rise to monocytes and numerous subsets of macrophages and DCs. Two major subsets of blood monocytes have been described in mice, humans, and other species. The two murine subsets, classical (which express Ly6C, recognized by the anti-Gr1 antibody) and nonclassical monocytes (Ly6Clo), are distinguished by differential expression of chemokine receptors, particularly CCR1, CCR2, and CX3CR1 (fractalkine receptor). Classical monocytes are known to exit the BM in a CCR2-dependent fashion and CCR2 ligands CCL2 (MCP-1) and CCL7 (MCP-3) help maintain homeostatic levels of monocytes in the circulation. In addition to the BM, the spleen is home to both subsets of mature monocytes which appear phenotypically similar to the two subsets found in blood.

Monocytes participate in tissue healing, clearance of pathogens and dead cells, and initiation of adaptive immunity. However, recruited monocytes can also contribute to the pathogenesis of infection and chronic inflammatory diseases and particularly may contribute to neuroinflammatory diseases. Related thereto it is known that during some insults to the brain, circulating monocytes can be mobilized to breach the BBB, migrate into the brain, and subsequently contribute to the neuroimmune response in association with microglia (M Prinz et al., “Heterogeneity of CNS myeloid cells and their roles in neurodegeneration”, Nat Neurosci 14, 1227-1235 (2011), Mildner, et al., “CCR2+Ly 6Chi monocytes are crucial for the effector phase of autoimmunity in the central nervous system”, Brain 132, 2487-2500 (2009)). Peripheral monocytes are also known to enter the brain after traumatic brain injury and contribute to neuronal injury (S Gyoneva, et al., “Ccr2 deletion dissociates cavity size and tau pathology after mild traumatic brain injury”, J Neuroinflammation 12, 228 (2015)), and they play a similar role in multiple sclerosis mouse models (Fife et al., “CC chemokine receptor 2 is critical for induction of experimental autoimmune encephalomyelitis”, J Exp Med 192, 899-905 (2000), Izikson et al., “Resistance to experimental autoimmune encephalomyelitis in mice lacking the CC chemokine receptor (CCR) 2”, J Exp Med 192, 1075-1080 (2000). In the experimental autoimmune encephalomyelitis (EAE) rodent model of multiple sclerosis, gene-expression profiles indicate that infiltrating monocytes are highly inflammatory compared with microglia. Moreover, invading monocytes induce axonal damage by initiating demyelination, whereas microglia clear debris (R Yamasaki, et al., “Differential roles of microglia and monocytes in the inflamed central nervous system”, J Exp Med 211, 1533-1549 (2014)).

It is further known that in models of trauma, neurological disease, or infection that inflammatory BM-derived monocytes traffic to and are recruited into inflamed tissue, including the brain and spinal cord (Donnelly, D. J., and Popovich, P. G. (2008), “Inflammation and its role in neuroprotection, axonal regeneration and functional recovery after spinal cord injury”, Exp. Neurol. 209, 378-388; Kigerl et al., (2009), “Identification of two distinct macrophage subsets with divergent effects causing either neurotoxicity or regeneration in the injured mouse spinal cord? J. Neurosci. 29, 13435-1344; McGavern, D. B., and Kang, S. S. (2011), “Illuminating viral infections in the nervous system”, Nat. Rev. Immunol. 11, 318-329). Further evidence also indicates that there is significant trafficking and recruitment of peripherally derived monocytes to the brain with psychological stress (Brevet et al. (2010), “Chronic foot-shock stress potentiates the influx of bone marrow-derived microglia into hippocampus”. J. Neurosci. Res. 88, 1890-1897; Wohleb et al., (2013) “Stress-Induced recruitment of bone marrow-derived monocytes to the brain promotes anxiety-like behavior”, J. Neurosci. 33, 13820-13833; Ataka, et al. (2013), “Bone marrow-derived microglia infiltrate into the paraventricular nucleus of chronic psychological stress-loaded mice”, PLOS ONE 8: e81744; Wohleb, E. S., Patterson, J. M., Sharma, V., Quan, N., Godbout, J. P., and Sheridan, J. F. (2014b), “Knockdown of interleukin-1 receptor type-1 on endothelial cells attenuated stress-induced neuroinflammation and prevented anxiety-like behavior”, J. Neurosci. 34, 2583-2591; Sawada, A., Nilyama, Y., Ataka, K., Nagaishi, K., Yamakage, M., and Fujimiya, M. (2014), “Suppression of bone marrow-derived microglia in the amygdala improves anxiety-like behavior induced by chronic partial sciatic nerve ligation in mice” Pain 155, 1762-1772). In these studies, monocytes traffic to the brain and differentiate into brain macrophages that promote inflammatory signaling and that trafficking of monocytes with stress and their accumulation in the brain can influence neuroinflammatory signaling and behavior (Terrando et al. (2011), “Resolving postoperative neuroinflammation and cognitive decline: Ann. Neurol. 70, 986-995; Wohleb et al. (2011), “β-Adrenergic receptor antagonism prevents anxiety-like behavior and microglial reactivity Induced by repeated social defeat” J. Neurosci. 31, 6277-6288; Wohleb, et al. (2014), “Re-establishment of anxiety in stress-sensitized mice is caused by monocyte trafficking from the spleen to the brain”, Biol. Psychiatry 75, 970-981; Beumer et al. (2012), “The immune theory of psychiatric diseases: a key role for activated microglia and circulating monocytes”, J. Leukoc. Biol. 92, 959-975; D'Mello, et al. (2013), “P-selectin-mediated monocyte-cerebral endothelium adhesive interactions link peripheral organ inflammation to sickness behaviors” J. Neurosci. 33, 14878-14888; Degos, et al. (2013), “Depletion of bone marrow-derived macrophages perturbs the innate immune response to surgery and reduces postoperative memory dysfunction”, Anesthesiology 118, 527-536; Sawada et al., (2014), “Suppression of bone marrow-derived microglia in the amygdala improves anxiety-like behavior induced by chronic partial sciatic nerve ligation in mice”, Pain 155, 1762-1772).

Accordingly, based on the foregoing methods and means for inhibiting M2 monocytes from being produced and/or inhibiting their trafficking/migration/infiltration to sites where they may cause adverse inflammation such as in the CNS are desired. Toward that end the present invention provides methods of alleviating and preventing the adverse side effects of M2 monocytes, particularly their involvement in causing or exacerbating chronic and inflammatory conditions, by preventing or inhibiting the differentiation of monocytes into the M2 phenotype and/or preventing or inhibiting the trafficking or migration of monocytes into inflammatory sites in a subject in need thereof by inhibiting/preventing the expression and/or function of BRI3 in the subject, particularly wherein the subject has or is at risk of developing a chronic or acute inflammatory condition, typically a chronic or acute neuroinflammatory condition.

### BRIEF DESCRIPTION OF THE FIGURES

FIG. 1A-D. Peripheral blood mononuclear cells (PBMCs) from five volunteers [n=2 healthy control (3401HC and 3402HC), 3 PD (PD134, 123, 126)] were subject to scRNASeq and the entire dataset was integrated to examine cell-type specific changes in gene expression in PD. Panel A contains a schematic representing the overall workflow from patient sampling to identification of differentially expressed genes in specific cell types (black arrows represent work flow), (UMAP: Uniform Manifold Approximation and Projection, SCINA: Semi-supervised Category Identification and Assignment, Muskat: Multi-sample multi-group scRNA-seq analysis tools). Panel B shows a table indicating the totals and percentages of various cell types in PD patients and controls. Panel C shows that the data integration pipeline successfully normalized the dataset to eliminate subject-driven variability. Panel D shows that no group-based variability was noted in the identification of clusters or assignment of cellular identity.

FIG. 2A-B contain experimental results relating to the identification of differentially expressed genes (DEGs) in monocytes. Panel A contains an MA plot demonstrating genes significantly altered in CD16 monocytes in PD [P value: 1.00e-11 (p_adj_loc)]. Panel B contains a Dot plot indicating percent detection (dot size) of DEGs split in order to compare PD patients (blue) to healthy controls (red). The data indicate that BRI3 expression levels are significantly elevated in PD (A), BRI3 expression is relatively restricted to monocytes (B), and that BRI3 is detectable in a higher percentage of PD patient monocytes compared to controls (B).

FIG. 3 contains a data summary of the results of experiments which determined the frequency and consistency of cells driving DEG findings. Volcano plots of selected genes demonstrating variation in individual patients and number of cells in which DEGs were detected are shown. The data shows that BRI3 elevation was consistently elevated in significant numbers of cells in all three PD patients sampled compared to control subjects (upper middle).

FIG. 4A-B contain experimental results wherein BRI3 was induced using inflammatory stimuli in a monocyte cell line. Panel A contains a Western blot demonstrating BRI3 upregulation and release following activation of cultured human THP-1 monocytes activated using sequential exposure to LPS and nigericin. Panel B shows the quantitation of independent western blot experiments using densitometry (n=3, P value >0.05, t-test).

FIG. 5 contains experimental results where directed differentiation of THP1 monocytes using standardized PMA priming and subsequent IL4/IL13 cytokine incubation drives the assumption of the M2/non-classical/pro-repair monocyte phenotype. Dat demonstrate that BRI3 expression is significantly elevated under conditions promoting the M2/non-classical/pro-repair phenotype.

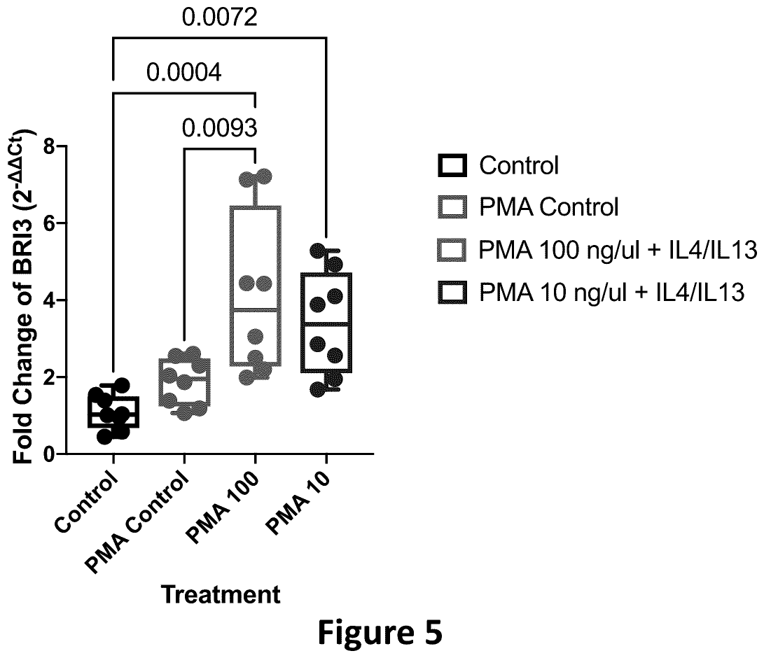

FIG. 6A-D contain experimental results demonstrating that the inactivation of BRI3 reduces cytokine expression. Panel A contains a Western blot analysis of four independent sgRNAs targeting BRI3. The information in the box depicts loss of BRI3 protein in cell lines derived using two independent sgRNAs (C and D). Panel B contains experimental results showing the confirmation of sequence alterations resulting from CRISPR/CAS9 inactivation of BRI3 genomic sequence in THP1 cells. Panel C and D contain experimental results showing the suppression of G-CSF (C) and IL2 (D) in THP-1 cells lacking BRI3 following sequential exposure to LPS and nigericin.

FIG. 7A-C contains experimental data demonstrating that BRI3 inactivation modifies monocyte phenotype. Four independently established control and BRI3 null THP-1 cultures were generated using CRISPR/CAS9. Cultures were differentiated using phorbol 12-myristate 13-acetate (PMA) and gene expression was evaluated using the nanostring platform. Panel A, and B contain data which show that pathway scoring identified an elevation in fatty acid synthesis score and a reduction in arginine metabolism score in normalized data from BRI3 inactivated cultures compared with control cultures. Panel C shows that reduced expression of CD84 was noted in BRI3 inactivated cultures compared with control (p=0.0010, one way ANOVA with Sidak's multiple comparisons test.

### OBJECTS OF THE INVENTION

It is an object of the invention to provide novel methods of preventing or inhibiting the differentiation of monocytes into the M2 phenotype and/or for preventing or inhibiting the trafficking or migration of monocytes into inflammatory sites in a subject in need thereof by inhibiting/preventing the expression and/or function of BRI3 in the subject.

It is a specific object of the invention to provide novel methods of preventing or inhibiting the differentiation of monocytes into the M2 phenotype and/or for preventing or inhibiting the trafficking or migration of monocytes into inflammatory sites in a subject in need thereof by inhibiting/preventing the expression and/or function of BRI3 in the subject wherein the subject has or is at risk of developing a chronic or acute inflammatory condition or wherein the subject has or is at risk of developing a chronic or acute neuroinflammatory condition.

In some exemplary embodiments the acute or chronic inflammatory condition is caused by an aging, infection, an environmental insult, an injury such as a repetitive motion injury, or an autoimmune or inflammatory condition.

In some exemplary embodiments the acute or chronic neuroinflammatory condition is caused by aging, an infection, an environmental insult, an injury such as a traumatic brain injury, an autoimmune condition, a sterile inflammatory condition, a demyelinating condition, stress, stroke or a neurodegenerative disease or other inflammatory condition that affects the central nervous system (CNS).

In some exemplary embodiments the subject is at risk of developing the inflammatory condition because of heredity, environmental exposure or an activity such as a sports activity such as football, boxing and the like where head trauma is likely to occur which correlates to increased risk of a neuroinflammatory condition.

In some exemplary embodiments the subject has an infection caused by a virus, bacteria, parasite, yeast or fungus which causes acute or chronic pathologic inflammation; e.g., acute or chronic pathologic neuroinflammation.

In some exemplary embodiments the subject has an infection caused by a coronavirus (e.g., SARS-COVID or SARS COVID 2 (Covid 19)), Japanese encephalitis virus, influenza virus, measles virus, herpes simplex virus, varicella zoster virus, mumps virus, chicken pox virus, rubella virus, Streptococcus pneumoniae, Mycoplasma pneumoniae, human metapneumovirus (HPMV), human parainfluenza virus, Legionnaires' disease, respiratory syncytial virus (RSV), rhinovirus, pneumocytitis pneumonia, or pneumococcal disease.

In some exemplary embodiments the subject has or is at risk of developing a neurodegenerative disease selected from Alzheimer's disease, Parkinson's disease (PD), senility or another memory disorder, ataxia, motor neuron disease, multiple sclerosis (MS), Lewy body disease or Lewy body dementia (LBD), multiple system atrophy (MSA), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)), optionally wherein the neurodegenerative diseases is PD.

In some exemplary embodiments the subject has or is at risk of developing a demyelinating condition is selected from multiple sclerosis, ALS, Transverse Myelitis, Guillain-Barre syndrome, Neuromyelitis optica, or Devic's disease, an Idiopathic inflammatory demyelinating disease, a Leukodystrophic or dysmyelinating disorder, Central pontine myelinolysis, a myelopathy such as tabes dorsalis (syphilitic myelopathy), Leukoencephalopathy such as progressive multifocal leukoencephalopathy, a Leukodystrophy, chronic inflammatory demyelinating polyneuropathy, anti-MAG peripheral neuropathy, Charcot-Marie-Tooth disease, Hereditary neuropathy and Progressive inflammatory neuropathy.

In some exemplary embodiments the subject has or is at risk of developing am autoimmune disease that affects the CNS is selected from Neuromyelitis optica, Anti-myelin oligodendrocyte glycoprotein antibody disease (MOG), Acute disseminated encephalomyelitis (ADEM), Chronic meningitis, Central nervous system (CNS) vasculitis, Hashimoto's encephalitis, Steroid responsive encephalopathy associated with autoimmune thyroiditis (SREAT), Neurosarcoidosis, Optic neuritis, or Transverse myelitis.

In some exemplary embodiments the subject has or is at risk of developing chronic or acute inflammation caused by a vaccine.

In some exemplary embodiments the subject has or is at risk of developing chronic or acute inflammation is caused by concussive injuries such as caused by a sports activity or by stroke or sepsis or acute respiratory disease syndrome (ARDS).

In some exemplary embodiments any of the previous methods further include administering an active agent that modifies the expression and/or function of FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof.

In some exemplary embodiments any of the previous methods further include detecting whether the expression and/or function of BRI3 or any of the previous genes recited is reduced by about 25%, about 30%, about 40%, about 45%, about 50%, about 55% about 60%, about 65%, about 70%, about 75%, about 80%, about 85%, about 90%, about 95%, or about 100% after treatment and/or increased by about 25%, about 30%, about 40%, about 45%, about 50%, about 55% about 60%, about 65%, about 70%, about 75%, about 80%, about 85%, about 90%, about 95%, or about 100% compared to normal levels prior to treatment.

In some exemplary embodiments any of the previous methods further include administering another active agent which increases the expression and/or function of one or more of HDDC2, EVL, UBB, RAC2, OAS1, LSP1, or ARF5, or any combination thereof, optionally wherein the increase is by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

In some exemplary embodiments in any of the previous methods BRI3 expression or activity after treatment is reduced in peripheral blood mononuclear cells (PBMCs), monocytes, dendritic cells, and/or central nervous system (CNS) cells, optionally wherein the monocytes are circulating monocytes, further optionally wherein the monocytes are CD16+ monocytes and/or CD14+ monocytes, and yet further optionally wherein the CNS cells comprise or are microglia.

In some exemplary embodiments the active agent reduces the expression and/or function of BRI3 in monocytes, optionally wherein the monocytes are circulating monocytes, further optionally wherein the monocytes are CD16+ monocytes CD14+ monocytes, and/or meningeal monocytes.

In some exemplary embodiments in any of the previous methods the active agent that modifies the expression and/or function of BRI3 or other gene comprises a clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′) 2), a chimeric antigen receptor (CAR)-expressing cell, a peptide, a small molecule, a polymer, an expression vector encoding a gene of interest, or any combination thereof.

In some exemplary embodiments in any of the previous methods the active agent which reduces BRI3 expression or activity:
(i) comprises a CRISPR/Cas gene editing agent against BRI3;
(ii) comprises or consists of a short-guide RNA (sgRNA) selected from the oligonucleotide sequences AACTCTATCGTGGTCGTAGG (SEQ ID NO:1), CGTCACAGGTGGGCCCGTAA (SEQ ID NO:2), GACTACGCGTGCGGCCCGCA (SEQ ID NO: 3) (depicted here in “sense” orientation),
(iii) comprises or consists of a sgRNA targeting BRI3 falling within or including any of the following genomic sequences:

(SEQ ID NO: 4)
GAGGAAGCGACGATGCCCCAACTGTGGAGC,
(SEQ ID NO: 5)
ACCACGATAGAGTTGGCAGGATAGCGGGTG,
(SEQ ID NO: 6)
AGTTGGGGCATCGTCGCTTCCTCAAGGCAA,
(SEQ ID NO: 7)
TTACGGGCCCACCTGTGACGAGGTAGGGGT,
(SEQ ID NO: 8)
GCCCTACCCCTACCTCGTCACAGGTGGGCC,
(SEQ ID NO: 9)
TCCTGCCAACTCTATCGTGGTCGTAGGAGG,
(SEQ ID NO: 10)
CCCGCTATCCTGCCAACTCTATCGTGGTCG,
(SEQ ID NO: 11)
GGGCGACTACGCGTGCGGCCCGCACGGCTA,
(SEQ ID NO: 12)
GCCCACCTGTGACGAGGTAGGGGTAGGGCG,
(SEQ ID NO: 13)
CCCAGGGTCTACAACATCCACAGCCGGACC,
(SEQ ID NO: 14)
CCACGATAGAGTTGGCAGGATAGCGGGTGA,
(SEQ ID NO: 15)
TTGGCAGGATAGCGGGTGACGGTCCGGCTG,
(SEQ ID NO: 16)
CAGGGATACCCACCCACCATCCCAGGGTCT,
(SEQ ID NO: 17)
CCTGGTGTTCCCTTTAAGCGAAGGTGGCTC

(as annotated in Gene ID 25798, NM_015379.5, or identical BRI3 sequences in prior or future annotations),
(iv) comprises a sgRNA sequence selected from one comprising or consisting of any of the following nucleic acid sequences:

(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 5)
ACCACGATAGAGTTGGCAGGATAGCGGGTG
(SEQ ID NO: 6)
AGTTGGGGCATCGTCGCTTCCTCAAGGCAA
(SEQ ID NO: 7)
TTACGGGCCCACCTGTGACGAGGTAGGGGT
(SEQ ID NO: 8)
GCCCTACCCCTACCTCGTCACAGGTGGGCC
(SEQ ID NO: 9)
TCCTGCCAACTCTATCGTGGTCGTAGGAGG
(SEQ ID NO: 10)
CCCGCTATCCTGCCAACTCTATCGTGGTCG
(SEQ ID NO: 11)
GGGCGACTACGCGTGCGGCCCGCACGGCTA
(SEQ ID NO: 12)
GCCCACCTGTGACGAGGTAGGGGTAGGGCG
(SEQ ID NO: 13)
CCCAGGGTCTACAACATCCACAGCCGGACC
(SEQ ID NO: 14)
CCACGATAGAGTTGGCAGGATAGCGGGTGA
(SEQ ID NO: 15)
TTGGCAGGATAGCGGGTGACGGTCCGGCTG
(SEQ ID NO: 16)
CAGGGATACCCACCCACCATCCCAGGGTCT
(SEQ ID NO: 17)
CCTGGTGTTCCCTTTAAGCGAAGGTGGCTC
(SEQ ID NO: 18)
AGCGGCCGCCCGCCTACAACCTGGAGGCCG
(SEQ ID NO: 19)
ACAGGGATACCCACCCACCATCCCAGGGTC
(SEQ ID NO: 20)
TAAGCGAAGGTGGCTCCACAGTTGGGGCAT
(SEQ ID NO: 21)
CCAAAGGGGAAGAGGATGATGGCCAGGAAG
(SEQ ID NO: 22)
TGGGATGGTGGGTGGGTATCCCTGTGGGCG
(SEQ ID NO: 23)
CAGCAAATGAACCCAAAGGGGAAGAGGATG
(SEQ ID NO: 24)
TACGGGCCCACCTGTGACGAGGTAGGGGTA
(SEQ ID NO: 25)
CTACGCGTGCGGCCCGCACGGCTACGGCGC
(SEQ ID NO: 26)
GGCGGGCGGCCGCTCCTGCAGCAGCGGCTT
(SEQ ID NO: 27)
GACCACAAGCCGCTGCTGCAGGAGCGGCCG
(SEQ ID NO: 28)
CTGCCCGTCTCTGCTGCAGGGTTGGGGTGC
(SEQ ID NO: 29)
CCCACCTGTGACGAGGTAGGGGTAGGGCGG
(SEQ ID NO: 30)
TGATGGCCAGGAAGATGCCCAGGAAGGTGA
(SEQ ID NO: 31)
GGGCCTGGTGTTCCCTTTAAGCGAAGGTGG
(SEQ ID NO: 32)
CTGTGGATGTTGTAGACCCTGGGATGGTGG
(SEQ ID NO: 33)
AGGCAAAACAGCAAATGAACCCAAAGGGGA
(SEQ ID NO: 34)
GCCGCCCTACCCCTACCTCGTCACAGGTGG
(SEQ ID NO: 35)
GCAAAACAGCAAATGAACCCAAAGGGGAAG
(SEQ ID NO: 36)
GTCGTAGGAGGCTGTCCTGTCTGCAGGTGA
(SEQ ID NO: 37)
GGCCGGCCAGGGCGACTACGCGTGCGGCCC
(SEQ ID NO: 38)
GGCCGCTCCTGCAGCAGCGGCTTGTGGTCC
(SEQ ID NO: 39)
GGCAAAACAGCAAATGAACCCAAAGGGGAA
(SEQ ID NO: 40)
CGCCTACAACCTGGAGGCCGGCCAGGGCGA
(SEQ ID NO: 41)
TGCGGGCCGCACGCGTAGTCGCCCTGGCCG
(SEQ ID NO: 42)
CCTGCAGCAGCGGCTTGTGGTCCATGGCGG
(SEQ ID NO: 43)
TCTGCTGCAGGGTTGGGGTGCTGGAGGACT
(SEQ ID NO: 44)
GCCGGCCTCCAGGTTGTAGGCGGGCGGCCG
(SEQ ID NO: 45)
CCGGCTGTGGATGTTGTAGACCCTGGGATG
(SEQ ID NO: 46)
CCATGGACCACAAGCCGCTGCTGCAGGAGC
(SEQ ID NO: 47)
TGTGACGAGGTAGGGGTAGGGCGGCGGCGG
(SEQ ID NO: 48)
TGGATGTTGTAGACCCTGGGATGGTGGGTG
(SEQ ID NO: 49)
AGGAGCGGCCGCCCGCCTACAACCTGGAGG
(SEQ ID NO: 50)
CTGGCCGGCCTCCAGGTTGTAGGCGGGCGG
(SEQ ID NO: 51)
GGATGTTGTAGACCCTGGGATGGTGGGTGG
(SEQ ID NO: 52)
ACGAGGTAGGGGTAGGGCGGCGGCGGGGGC
(SEQ ID NO: 53)
AGGATGATGGCCAGGAAGATGCCCAGGAAG
(SEQ ID NO: 54)
CTCTGCCCGTCTCTGCTGCAGGGTTGGGGT
(SEQ ID NO: 55)
GACGAGGTAGGGGTAGGGCGGCGGCGGGGG
(SEQ ID NO: 56)
GTTGTAGACCCTGGGATGGTGGGGGGTAT
(SEQ ID NO: 57)
GGCGGGGATGGCGCCGTAGCCGTGCGGGCC
(SEQ ID NO: 58)
GGGTTCATTTGCTGTTTTGCCTTGAGGAAG
(SEQ ID NO: 59)
CGAGGTAGGGGTAGGGCGGCGGCGGGGGCG
(SEQ ID NO: 60)
CCTGGCCGGCCTCCAGGTTGTAGGCGGGCG
(SEQ ID NO: 61)
CTGGCCATCATCCTCTTCCCCTTTGGGTTC
(SEQ ID NO: 62)
TGAACCCAAAGGGGAAGAGGATGATGGCCA
(SEQ ID NO: 63)
GAGGTAGGGGTAGGGCGGCGGCGGGGGCGC
(SEQ ID NO: 64)
GCCGCCCGCCTACAACCTGGAGGCCGGCCA
(SEQ ID NO: 65)
GGCCGCACGCGTAGTCGCCCTGGCCGGCCT
(SEQ ID NO: 66)
TGTTGTAGACCCTGGGATGGTGGGTGGGTA
(SEQ ID NO: 67)
GGATAGCGGGTGACGGTCCGGCTGTGGATG
(SEQ ID NO: 68)
AACTGTGGAGCCACCTTCGCTTAAAGGGAA
(SEQ ID NO: 69)
CCTGGCCATCATCCTCTTCCCCTTTGGGTT
(SEQ ID NO: 70)
TTAAGCGAAGGTGGCTCCACAGTTGGGGCA
(SEQ ID NO: 71)
TCTGCCCGTCTCTGCTGCAGGGTTGGGGTG
(SEQ ID NO: 72)
ACTGTGGAGCCACCTTCGCTTAAAGGGAAC
(SEQ ID NO: 73)
TTTAAGCGAAGGTGGCTCCACAGTTGGGGC
(SEQ ID NO: 74)
GCGGGGATGGCGCCGTAGCCGTGCGGGCCG
(SEQ ID NO: 75)
CGCCCTGGCCGGCCTCCAGGTTGTAGGCGG
(SEQ ID NO: 76)
TCCGGCTGTGGATGTTGTAGACCCTGGGAT
(SEQ ID NO: 77)
GCGTAGTCGCCCTGGCCGGCCTCCAGGTTG
(SEQ ID NO: 78)
CCGCCTACAACCTGGAGGCCGGCCAGGGCG
(SEQ ID NO: 79)
GCTGGAGGACTGCTTCACCTTCCTGGGCAT
(SEQ ID NO: 80)
GCTTCACCTTCCTGGGCATCTTCCTGGCCA
(SEQ ID NO: 81)
GCGGCGGCGGGGGCGCGGCGGGGATGGCGC
(SEQ ID NO: 82)
GTCTCTGCTGCAGGGTTGGGGTGCTGGAGG
(SEQ ID NO: 83)
TAGGGCGGCGGCGGGGGCGCGGCGGGGATG
(SEQ ID NO: 84)
TGCTGGAGGACTGCTTCACCTTCCTGGGCA
(SEQ ID NO: 85)
GGTAGGGCGGCGGCGGGGGCGCGGCGGGGA
(SEQ ID NO: 86)
AGGGGTAGGGCGGCGGCGGGGGCGCGGCGG
(SEQ ID NO: 87)
GTAGGGCGGCGGCGGGGGCGCGGCGGGGAT;

or
(v) comprises any anti-BRI3 agent utilizing CRISPR/Cas9 with an inactivated endonuclease, referred to as “dead” Cas9 or “dCas9.”

In some exemplary embodiments in any of the previous methods the active agent which reduces BRI3 expression or activity comprises or consists of one or more short-guide RNAs having sequences selected from one or more of oligonucleotide sequences AACTCTATCGTGGTCGTAGG (SEQ ID NO:1), CGTCACAGGTGGGCCCGTAA (SEQ ID NO:2), GACTACGCGTGCGGCCCGCA (SEQ ID NO:3) (depicted here in “sense” orientation) and optionally inactivated endonuclease, further optionally “dead” Cas9 or “dCas9.”

In some exemplary embodiments any of the previous methods further comprises administering at least one other active agent, optionally wherein the at least one other active agent is levodopa, carbidopa, a dopamine agonist (e.g., pramipexole, ropinirole, rotigotine, apomorphine), a monoamine oxidase B (MAO B) inhibitor (e.g., selegiline, rasagiline, safinamide), a catechol O-methyltrasnferase (COMT) inhibitor (e.g., entacapone, tolcapone), an anticholinergic (e.g., benztropine), or amantadine, or any combination thereof, further optionally comprising administering deep brain stimulation (DBS).

In some exemplary embodiments any of the previous methods further comprises conducting a treatment which effects one or more of the following:
(i) increasing B cells;
(ii) increasing dendritic cells; or
(iii) reducing CD4⁺ T cells,
   optionally wherein the increasing and/or reducing in any one or more of (i)-(iii) at least takes place in PBMCs.

In some exemplary embodiments in any of the previous methods further comprises detecting the expression and/or function of BRI3 in monocytes of the subject, and optionally one or more of FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, wherein said detecting occurs prior, during and/or after said administrating, optionally wherein the detecting comprises detecting in one or more samples from the treated subject, optionally a blood sample and/or a CNS or brain sample.

In some exemplary embodiments in any of the previous methods further comprises determining whether the severity and/or stage of and/or inflammation, optionally associated with a neurodegenerative disease has decreased or increased in a subject before, after or during treatment, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject at a first time point and a second time point, and optionally measuring the quantity (number and/or percentage) of CD16+ monocytes, B cells, dendritic cells, CD14+ monocytes, and CD4+ T cells in the subject or in the sample at the first time point and the second time point; and
(b) determining that the severity and/or stage and/or inflammation has decreased or increased
   if:
      (i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is lower or higher at the second time point than the first time point; and/or
      (ii) the expression of HDDC2, EVL, UBB, RAC2, OAS1, LSP1, or ARF5, or any combination thereof is higher or lower at the second time point than the first time point,
   and optionally if:
      (iii) the quantity of CD16+ monocytes, B cells, and/or dendritic cells is lower at the second time point than the first time point; and/or
      (iv) the quantity of CD14+ monocytes and/or CD4⁺ T cells is higher at the second time point than the first time point,
         optionally wherein the neurodegenerative disease is PD,
         further optionally wherein the method comprises one or more of the following:
(I) in (a), at least the expression of BRI3 is measured;
(II) in (b), at least the expression of BRI3 is higher at the second time point than the first time point;
(III) in (b) (i), the expression is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(IV) in (b) (ii), the expression is lower at the second time point than the first time point by about 25%, about 30%, about 40%, about 45%, about 50%, about 55% about 60%, about 65%, about 70%, about 75%, about 80%, about 85%, about 90%, about 95%, or about 100%;
(V) in (b) (iii), the quantity is lower at the second time point than the first time point by about 25%, about 30%, about 40%, about 45%, about 50%, about 55% about 60%, about 65%, about 70%, about 75%, about 80%, about 85%, about 90%, about 95%, or about 100%; and/or
(VI) in (b) (iv), the quantity is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

In some exemplary embodiments the methods include determining whether the method is effective in a subject, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject before and at one or more time points after starting the therapy, and optionally measuring the quantity (number and/or percentage) of CD16+ monocytes, B cells, dendritic cells, CD14+ monocytes, and CD4+ T cells in the subject or in the sample before and at one or more time points after starting the therapy; and
(b) determining that the therapy is effective
   if:
      (i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is lower at least one time point after starting the therapy compared to before starting the therapy; and/or
      (ii) the expression of HDDC2, EVL, UBB, RAC2, OAS1, LSP1, or ARF5, or any combination thereof is higher at least one time point after starting the therapy compared to before starting the therapy,
   and optionally if:
      (iii) the quantity of CD16+ monocytes, B cells, and/or dendritic cells is lower at least one time point after starting the therapy compared to before starting the therapy; and/or
      (iv) the quantity of CD14+ monocytes and/or CD4⁺ T cells is higher least one time point after starting the therapy compared to before starting the therapy,
         optionally wherein the neurodegenerative disease is PD,
         further optionally wherein the method comprises one or more of the following:
(I) in (a), at least the expression of BRI3 is measured;
(II) in (b), at least the expression of BRI3 is lower at least one time point after starting the therapy compared to before starting the therapy;
(III) in (b) (i), the expression is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%.

In some exemplary embodiments in any of the previous methods BRI3 or other gene detection measuring is effected in the subject or a sample isolated from the subject by one or more of RNA sequencing, RNA-sequencing at single cell resolution, DNA array, flow cytometry, histochemistry, protein detection (optionally by use of bead-based or solid phase protein detection methods, further optionally wherein protein detection is effected by the use of one or more of an immunosorbent assay, gel electrophoresis, SDS-PAGE (polyacrylamide gel electrophoresis), Liquid chromatography-mass spectrometry (LC-MS), HPLC, ELISA, immunoelectrophoresis, immunostaining, Western blot, protein colorimetric assay, flow cytometry, electron microscopy, an enzyme assay, immune fluorescence, spectrophotometry, and the like) or imaging, optionally wherein the sample comprises PBMCs, monocytes, dendritic cells, and/or central nervous system (CNS) cells, optionally wherein the monocytes are circulating monocytes, further optionally wherein the monocytes are CD16⁺ monocytes, CD14⁺ monocytes, and/or meningeal monocytes, and yet further optionally wherein the CNS cells comprise microglia.

In some exemplary embodiments any of the previous methods further include the administration of an anti-inflammatory agent such as a steroid, corticosteroid, an NSAID such as buprofen, Naproxen, diclofenac, celecoxib, mefenamic acid, etoricoxib, indomethacin or high dose aspirin, a biologic such as IL-6 or TNF antagonist antibody, Embrel, Humira, Actarit, Adelmidrol, Allicin, Amixetrine, Amlexanox, Azerizin, Baricitinib, BMS-345541, BMS-470539, Bufexamac, Cannabichromene, Cannabidiol, Cepharanthine, Cyclopentenone prostaglandin, Dagrocorat, Dapansutrile, Diacerein, EF-24, Enterococcus durans, Epiestriol, Fluasterone, Fosdagrocorat, Hinokinin, Ibuproxam, Icatibant, Ligstroside, Lisofylline, Mapracorat, Meconopsis horridula, Mesalazine, Minocycline, Modafinil, Mofezolac, Nangibotide, NR58-3.14.3, Oleocanthal, Oleopicrin, Oleuropein, Omega-3 fatty acid, Pregnenolone, Prostaglandin inhibitor, Pseudopterosin E, Safotibant, Selective glucocorticoid receptor modulator, Semapimod, Shea butter, a Statin, Tetramethylpyrazine, Toreforant, Trofinetide, Upadacitinib, Vinyldithiin, or VUF-600 or any combination of the foregoing.

### DETAILED DESCRIPTION OF THE INVENTION

### Definitions

Although various embodiments and examples of the present invention have been described referring to certain molecules, compositions, methods, or protocols, it is to be understood that the present invention is not limited to the particular molecules, compositions, methods, or protocols described herein, as theses may vary. It is also to be understood that the terminology used in the description is for the purpose of describing the particular versions or embodiments only and is not intended to limit the scope of the present invention which will be limited only by the appended claims.

All publications mentioned herein are incorporated herein by reference. Nothing herein is to be construed as an admission that the present invention is not entitled to antedate such disclosure by virtue of prior invention.

In the specification above and in the appended claims, all transitional phrases such as “comprising,” “including,” “having,” “containing,” “involving,” “composed of,” and the like are to be understood to be open-ended, namely, to mean including but not limited to. Only the transitional phrases “consisting of” and “consisting essentially of” shall be closed or semi-closed transitional phrases, respectively.

It must also be noted that, unless the context clearly dictates otherwise, the singular forms “a,” “an,” and “the” as used herein and in the appended claims include plural reference. Thus, the reference to “a cell” refers to one or more cells and equivalents thereof known to those skilled in the art, and so forth. Unless defined otherwise, all technical and scientific terms used herein have the same meanings as commonly understood by a person of skilled in the art.

It should be understood that, unless clearly indicated otherwise, in any methods disclosed or claimed herein that comprise more than one step, the order of the steps to be performed is not restricted by the order of the steps cited.

The term “about” or “approximately” as used herein when referring to a numerical value, such as of weight, mass, volume, concentration, or time, should not be limited to the recited numerical value but rather encompasses variations of +/−10% of a given value.

“BRI3” as used herein, also known as “brain protein I3,” “I3” or “pRGR2”, is in human encoded by the BRI3 gene in Chromosome 7 at 7q21.3 (Gene ID: 25798). Three BRI protein family members have been identified including BRI3 and BRI2 The latter reportedly is associated with Familial Danish and Familial British dementias. ‘In silico’ sequence analysis identified putative PP1 binding sites in BRI2 and BRI3. Protein phosphorylation is a major mechanism regulating intracellular processes. Protein phosphatase 1 (PP1) interacting proteins (PIPs) are fundamental in determining substrate specificity and subcellular localization of this phosphatase. More than 200 PIPs have thus far been reported. It has been shown that BRI2 and BRI3 bind PP1. The subcellular distribution of BRI2 and BRI3 is similar; both localize to the perinuclear area and Golgi apparatus in non-neuronal cells. However, in SH-SY5Y cells, BRI2 and BRI3 could also be detected in elongated cellular projections (‘processes’) and in rat cortical neurons both are broadly distributed throughout the cell body, neuritis and the nucleus. Co-localization of BRI2 and BRI3 with PP1 has been shown. The functional significance of these complexes has not been established. Interestingly, the Alzheimer's amyloid precursor protein (APP), forms a trimeric complex composed of PP1 and Fe65, with PP1 having the capacity to dephosphorylate APP at Thr668 residue. It has been suggested that PP1 containing complexes may be involved in regulating signaling events underlying neuropathological conditions.

An “anti-BRI3 agent” as used herein refers to any agents that are able to target BRI3 directly or indirectly. Anti-BRI3 agents of the present invention include, but are not limited to, any single guide RNA sequence targeting BRI3 through clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′)2), a chimeric antigen receptor (CAR)-expressing cell, a peptide, a small molecule, a polymer, an expression vector encoding a gene of interest, or any combination thereof. In some exemplary embodiments, the anti-BRI3 agent may comprise (i) short-guide RNA including but not limited to oligonucleotide sequences AACTCTATCGTGGTCGTAGG (SEQ ID NO:1), CGTCACAGGTGGGCCCGTAA (SEQ ID NO:2), GACTACGCGTGCGGCCCGCA (SEQ ID NO:3) (depicted here in “sense” orientation) or may comprise (ii) CRISPR/Cas9 with an inactivated endonuclease, referred to as “dead” Cas9 or “dCas9.”

The term “antibody” or “Ab,” or “immunoglobulin” is used herein in the broadest sense and encompasses various antibody structures which specifically binds with an antigen, including but not limited to monoclonal antibodies, polyclonal antibodies, multispecific antibodies (e.g., bispecific antibodies), and/or antibody fragments (also referred to as “antigen-binding antibody fragments”). Typically, a full-size Ab (also referred to as an intact Ab) comprises two pairs of chains, each pair comprising a heavy chain (HC) and a light chain (LC). A HC typically comprises a variable region and a constant region. A LC also typically comprises a variable region and constant region. The variable region of a heavy chain (VH) typically comprises three complementarity-determining regions (CDRs), which are referred to herein as CDR 1, CDR 2, and CDR 3 (or referred to as CDR-H1, CDR-H2, CDR-H3, respectively). The constant region of a HC typically comprises a fragment crystallizable region (Fc region), which dictates the isotype of the Ab, the type of Fc receptor the Ab binds to, and therefore the effector function of the Ab. Any isotype, such as IgG1, IgG2a, IgG2b, IgG3, IgG4, IgM, IgD, IgE, IgGA1, or IgGA2, may be used. Fc receptor types include, but are not limited to, FcaR (such as FcaRI), Fca/mR, FceR (such as FceRI, FceRII), FcgR (such as FcgRI, FcgRIIA, FcgRIIB1, FcgRIIB2, FcgRIIIA, FcgRIIIB), and FcRn and their associated downstream effects are well known in the art. The variable region of a light chain (VL) also typically comprises CDRs, which are CDR 1, CDR 2, and CDR 3 (or referred to as CDR-L1, CDR-L2, CDR-L3, respectively). In some embodiments, the antigen is ACVR1C (also referred to as ALK7). Antibodies can be intact immunoglobulins derived from natural sources or from recombinant sources. A portion of an antibody that comprises a structure that enables specific binding to an antigen is referred to “antigen-binding fragment,” “AB domain,” “antigen-binding region,” or “AB region” of the Ab.

Certain amino acid modifications in the Fc region are known to modulate Ab effector functions and properties, such as, but not limited to, antibody-dependent cellular cytotoxicity (ADCC), antibody-dependent cellular phagocytosis (ADCP), complement dependent cytotoxicity (CDC), and half-life (Wang X. et al., Protein Cell. 2018 January; 9 (1): 63-73; Dall'Acqua W. F. et al., J Biol Chem. 2006 Aug. 18; 281 (33): 23514-24. Epub 2006 Jun. 21; Monnet C. et al, Front Immunol. 2015 Feb. 4; 6:39. doi: 10.3389/fimmu.2015.00039. eCollection 2015). The mutation may be symmetrical or asymmetrical. In certain cases, antibodies with Fc regions that have asymmetrical mutation(s) (i.e., two Fc regions are not identical) may provide better functions such as ADCC (Liu Z. et al. J Biol Chem. 2014 Feb. 7; 289 (6): 3571-3590).

An IgG1-type Fc optionally may comprise one or more amino acid substitutions. Such substitutions may include, for example, N297A, N297Q, D265A, L234A, L235A, C226S, C229S, P238S, E233P, L234V, G236-deleted, P238A, A327Q, A327G, P329A, K322A, L234F, L235E, P331S, T394D, A330L, P331S, F243L, R292P, Y300L, V3051, P396L, S239D, 1332E, S298A, E333A, K334A, L234Y, L235Q, G236W, S239M, H268D, D270E, K326D, A330M, K334E, G236A, K326W, S239D, E333S, S267E, H268F, S324T, E345R, E430G, S440Y, M428L, N434S, L328F, M252Y, S254T, T256E, and/or any combination thereof (the residue numbering is according to the EU index as in Kabat) (Dall'Acqua W. F. et al., J Biol Chem. 2006 Aug. 18; 281 (33): 23514-24. Epub 2006 Jun. 21; Wang X. et al., Protein Cell. 2018 January; 9 (1): 63-73), or for example, N434A, Q438R, S440E, L432D, N434L, and/or any combination thereof (the residue numbering according to EU numbering). The Fc region may further comprise one or more additional amino acid substitutions. Such substitutions may include but are not limited to A330L, L234F, L235E, P3318, and/or any combination thereof (the residue numbering is according to the EU index as in Kabat). Specific exemplary substitution combinations for an IgG1-type Fc include, but not limited to: M252Y, S254T, and T256E (“YTE” variant); M428L and N434A (“LA” variant), M428L and N434S (“LS” variant); M428L, N434A, Q438R, and S440E (“LA-RE” variant); L432D and N434L (“DEL” variant); and L234A, L235A, L432D, and N434L (“LALA-DEL” variant) (the residue numbering is according to the EU index as in Kabat). In particular embodiments, an IgG1-type Fc variant may comprise the amino acid sequence of SEQ ID NOS: 11, 12, 13, 14, 15, 16, or 17.

When the Ab is an IgG2, the Fc region optionally may comprise one or more amino acid substitutions. Such substitutions may include but are not limited to P238S, V234A, G237A, H268A, H268Q, H268E, V309L, N297A, N297Q, A330S, P331S, C232S, C233S, M252Y, S254T, T256E, and/or any combination thereof (the residue numbering is according to the EU index as in Kabat). The Fc region optionally may further comprise one or more additional amino acid substitutions. Such substitutions may include but are not limited to M252Y, S254T, T256E, and/or any combination thereof (the residue numbering is according to the EU index as in Kabat).

An IgG3-type Fc region optionally may comprise one or more amino acid substitutions. Such substitutions may include but are not limited to E235Y (the residue numbering is according to the EU index as in Kabat).

An IgG4-type Fc region optionally may comprise one or more amino acid substitutions. Such substitutions may include but are not limited to, E233P, F234V, L235A, G237A, E318A, S228P, L236E, S241P, L248E, T394D, M252Y, S254T, T256E, N297A, N297Q, and/or any combination thereof (the residue numbering is according to the EU index as in Kabat). The substitution may be, for example, S228P (the residue numbering is according to the EU index as in Kabat).

In some cases, the glycan of the human-like Fc region may be engineered to modify the effector function (for example, see Li T. et al., Proc Natl Acad Sci USA. 2017 Mar. 28; 114 (13): 3485-3490. doi: 10.1073/pnas.1702173114. Epub 2017 Mar. 13).

The term “antibody fragment” or “Ab fragment” as used herein refers to any portion or fragment of an Ab, including intact or full-length Abs that may be of any class or sub-class, including IgG and sub-classes thereof, IgM, IgE, IgA, and IgD. The term encompasses molecules constructed using one or more potions or fragments of one or more Abs. An Ab fragment can be immunoreactive portions of intact immunoglobulins. The term is used in the broadest sense and includes polyclonal and monoclonal antibodies, including intact antibodies and functional (antigen-binding) antibody fragments, including fragment antigen binding (Fab) fragments, F(ab′)2 fragments, Fab′ fragments, Fv fragments, recombinant IgG (rIgG) fragments, single chain antibody fragments, including single chain variable fragments (scFv), diabodies, and single domain antibodies (e.g., sdAb, sdFv, nanobody) fragments. The term also encompasses genetically engineered and/or otherwise modified forms of immunoglobulins, such as intrabodies, peptibodies, chimeric antibodies, fully human antibodies, humanized antibodies, and heteroconjugate antibodies, multispecific, e.g., bispecific, antibodies, diabodies, triabodies, and tetrabodies, tandem di-scFv, tandem tri-scFv. In a specific embodiment, the antibody fragment is a scFv. Unless otherwise stated, the term “Ab fragment” should be understood to encompass functional antibody fragments thereof. A portion of an Ab fragment that comprises a structure that enables specific binding to an antigen is referred to as “antigen-binding Ab fragment,” “AB domain,” “antigen-binding region,” or “antigen-binding region” of the Ab fragment.

An “isolated” biological component (such as an isolated protein, nucleic acid, vector, or cell) refers to a component that has been substantially separated or purified away from its environment or other biological components in the cell of the organism in which the component naturally occurs, for instance, other chromosomal and extra-chromosomal DNA and RNA, proteins, and organelles. Nucleic acids and proteins that have been “isolated” include nucleic acids and proteins purified by standard purification methods. The term also embraces nucleic acids and proteins prepared by recombinant technology as well as chemical synthesis. An isolated nucleic acid or protein can exist in substantially purified form, or can exist in a non-native environment such as, for example, a host cell.

The term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes).

The term “macrophage” refers to Macrophages

The term “macrophage” refers to a type of white blood cell of the immune system that engulfs and digests anything lacking on its surface, proteins that are specific to healthy body cells, including cancer cells, microbes, cellular debris, foreign substances, etc. The process is called phagocytosis, which acts to defend the host against infection and injury. Phagocytes are found in essentially all tissues, where they patrol for potential pathogens by amoeboid movement. They take various forms (with various names) throughout the body (e.g., histiocytes, Kupffer cells, alveolar macrophages, microglia, and others), but all are part of the mononuclear phagocyte system. Besides phagocytosis, they play a critical role in nonspecific defense (innate immunity) and also help initiate specific defense mechanisms (adaptive immunity) by recruiting other immune cells such as lymphocytes. For example, they are important as antigen presenters to T cells. In humans, dysfunctional macrophages cause severe diseases such as chronic granulomatous disease that result in frequent infections. They are also suspected to be important in the formation of important organs like the heart and brain.

The term “monocyte” refers to type of leukocyte, or white blood cell. They are the largest type of leukocyte and can differentiate into macrophages and conventional dendritic cells. As a part of the vertebrate innate immune system monocytes also influence the process of adaptive immunity. There are at least three subclasses of monocytes in human blood based on their phenotypic receptors including M1 and M2 monocytes. Monocytes are produced by the bone marrow from precursors called monoblasts, bipotent cells that differentiated from hematopoietic stem cells. Monocytes circulate in the bloodstream for about one to three days and then typically move into tissues throughout the body where they differentiate into macrophages and dendritic cells. They constitute between three and eight percent of the leukocytes in the blood. About half of the body's monocytes are stored as a reserve in the spleen in clusters in the red pulp's Cords of Billroth.

The term “M1 monocyte” refers to monocytes which are positive for CD14, CD68, and CCR2 monocytes.

The term “M2 monocyte” refers to monocytes which are positive for CD14, CX3CR1, and CD163 or CD206.

“Neurodegenerative diseases” are a heterogeneous group of disorders that are characterized by the progressive degeneration of the structure and function of the central nervous system or peripheral nervous system. Exemplary neurodegenerative diseases include but are not limited to Parkinson's disease (PD), Alzheimer's disease (AD), multiple sclerosis (MS), Lewy body disease or Lewy body dementia (LBD), progressive supranuclear palsy (PSP), multiple system atrophy (MSA), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), and prion disease such as Creutzfeldt-Jakob disease (CJD).

The term “nucleic acid” and “polynucleotide” refer to RNA or DNA that is linear or branched, single or double stranded, or a hybrid thereof. The term also encompasses RNA/DNA hybrids. The following are non-limiting examples of polynucleotides: a gene or gene fragment, exons, introns, mRNA, tRNA, rRNA, ribozymes, cDNA, recombinant polynucleotides, branched polynucleotides, plasmids, vectors, isolated DNA of any sequence, isolated RNA of any sequence, nucleic acid probes and primers. A polynucleotide may comprise modified nucleotides, such as methylated nucleotides and nucleotide analogs, uracil, other sugars and linking groups such as fluororibose and thiolate, and nucleotide branches. The sequence of nucleotides may be further modified after polymerization, such as by conjugation, with a labeling component. Other types of modifications included in this definition are caps, substitution of one or more of the naturally occurring nucleotides with an analog, and introduction of means for attaching the polynucleotide to proteins, metal ions, labeling components, other polynucleotides or solid support. The polynucleotides can be obtained by chemical synthesis or derived from a microorganism. The term “gene” is used broadly to refer to any segment of polynucleotide associated with a biological function. Thus, genes include introns and exons as in genomic sequence, or just the coding sequences as in cDNAs and/or the regulatory sequences required for their expression. For example, gene also refers to a nucleic acid fragment that expresses mRNA or functional RNA, or encodes a specific protein, and which includes regulatory sequences.

The term “pharmaceutically acceptable excipient,” “pharmaceutical excipient,” “excipient,” “pharmaceutically acceptable carrier,” “pharmaceutical carrier,” or “carrier” as used herein refers to compounds or materials conventionally used in pharmaceutical compositions during formulation and/or to permit storage. Excipients included in the formulations will have different purposes. Examples of generally used excipients include, without limitation: saline, buffered saline, dextrose, water-for-infection, glycerol, ethanol, and combinations thereof, stabilizing agents, solubilizing agents and surfactants, buffers and preservatives, tonicity agents, bulking agents, and lubricating agents.

The term “recombinant” means a polynucleotide, a protein, a cell, and so forth with semi-synthetic or synthetic origin which either does not occur in nature or is linked to another polynucleotide, a protein, a cell, and so forth in an arrangement not found in nature.

The term “subject” as used herein may be any living organisms, preferably a mammal. In some embodiments, the subject is a primate such as a human. In some embodiments, the primate is a monkey or an ape. The subject can be male or female and can be any suitable age, including infant, juvenile, adolescent, adult, and geriatric subjects. In some examples, the patient or subject is a validated animal model for disease and/or for assessing toxic outcomes. The subject may also be referred to as “patient” in the art. The subject may have a disease or may be healthy.

The term “subject at increased risk of developing PD (or other neurologic disease associated with neural inflammation)” as used herein may be any living organism, preferably a mammal, and most preferably a primate such as a human that is at increased risk of developing PD (or other neurologic disease associated with neural inflammation) because of a family history, environmental factors, one or more concussions among other risk factors.

The term “subject having PD (or other neurologic disease associated with neural inflammation)” or “a subject determined to be at increased risk of developing PD (or other neurologic disease associated with neural inflammation)” as used herein may be any living organism, preferably a mammal, and most preferably a primate such as a human that has or is at increased risk of developing PD (or other neurologic disease associated with neural inflammation) who is determined to be at increased risk of developing PD (or other neurologic disease associated with neural inflammation) because of the increased or decreased expression of a gene the expression of which correlates with PD (or other neurologic disease associated with neural inflammation), e.g., increased expression of BRI3 on monocytes compared to BRI3 expression by normal controls.

The term “scFv,” “single-chain Fv,” or “single-chain variable fragment” refers to a fusion protein comprising at least one antibody fragment comprising a variable region of a light chain and at least one antibody fragment comprising a variable region of a heavy chain, wherein the light and heavy chain variable regions are contiguously linked, e.g., via a synthetic linker, e.g., a short flexible polypeptide linker, and capable of being expressed as a single chain polypeptide, and wherein the scFv retains the specificity of the intact antibody from which it is derived. Unless specified, as used herein an scFv may have the VL and VH variable regions in either order, e.g., with respect to the N-terminal and C-terminal ends of the polypeptide, the scFv may comprise VL-linker-VH or may comprise VH-linker-VL. The linker may comprise portions of the framework sequences. In scFvs, the heavy chain variable domain (HC V, HCV, or VH) may be placed upstream of the light chain variable domain (LC V, LCV, or VL), and the two domains may optionally be linked via a linker (for example, the G4S X3 linker). Alternatively, the heavy chain variable domain may be placed downstream of the light chain variable domain, and the two domains may optionally be linked via a linker (for example, the G4S X3 linker).

As used herein, the term “treat,” “treatment,” or “treating” generally refers to the clinical procedure for reducing or ameliorating the progression, severity, and/or duration of a disease or of a condition, or for ameliorating one or more conditions or symptoms (preferably, one or more discernible ones) of a disease. The disease to be treated may be, for example, PD, but may also treat other neurodegenerative diseases that cause a similar condition and/or symptom to that of PD. Therefore, the treatment method according to the present disclosure may also treat, a fibrotic disease, for example, pulmonary fibrosis, an interstitial lung disease, cystic fibrosis, chronic obstructive pulmonary disease, sarcoidosis, an allergic airway disease, hepatic fibrosis, or cardiac fibrosis. The condition to be treated by a method according to the present invention may be, for example, fibrosis, oxidative stress, or inflammation. In specific embodiments, the effect of the “treatment” may be evaluated by the amelioration of at least one measurable physical parameter of a disease, resulting from the administration of one or more therapies (e.g., anti-BRI3 agent, and in some cases in combination with another therapy). The parameter may be, for example, gene expression profiles, the mass of disease-affected tissues, inflammation-associated markers, fibrosis-associated markers, the number or frequency of disease-associated cells, the presence or absence of certain cytokines or chemokines or other disease-associated molecules, and may not necessarily discernible by the patient. In other embodiments “treat”, “treatment,” or “treating” may result in the inhibition of the progression of a disease, either physically by, e.g., stabilization of a discernible symptom, physiologically by, e.g., stabilization of a physical parameter, or both. In other embodiments the terms “treat”, “treatment” and “treating” refer to the reduction or stabilization of inflammatory or fibrotic tissue. Additionally, the terms “treat,” and “prevent” as well as words stemming therefrom, as used herein, do not necessarily imply 100% or complete cure or prevention. Rather, there are varying degrees of treatment effects or prevention effects of which one of ordinary skill in the art recognizes as having a potential benefit or therapeutic effect. In this respect, the inventive methods can provide any amount of any level of treatment or prevention effects of a disease in a mammal. Furthermore, the treatment or prevention provided by the inventive method can include treatment or prevention of one or more conditions or symptoms of the disease being treated or prevented. Also, for purposes herein, “prevention” can encompass delaying the onset of the disease, or a symptom or condition thereof.

The present invention relates to preventing or inhibiting the production of M2 monocytes and/or preventing or inhibiting monocyte trafficking, particularly M2 monocytes, e.g., to inflamed sites, e.g., in the central nervous system in a subject in need thereof, e.g., a subject having or at risk of developing a neuroinflammatory condition by inhibiting the expression or activity of “BRI3” or “brain protein 13”. As shown infra, we conducted a large-scale transcriptomic analysis of blood cells obtained from Parkinson's disease (PD) patients at single cell resolution. We have shown that BRI3 is robustly elevated in CD16 or M2 monocytes in Parkinson's disease (PD) patients. CD16 expression indicates either an intermediate or non-classical “M2” monocyte state with tissue invasion, pro-inflammatory, and anti-inflammatory potential.

We inactivated BRI3 in cultured monocytes and conducted transcriptomics using the Nanostring platform. We analyzed four independently derived BRI3 null lines generated using the CRISPR/CAS9 reagents described herein. We found that BRI3 modulates monocyte gene expression following PMA directed differentiation into the CD16 phenotype. Also, we identified significant alterations in genes required for phenotypic changes related to the M2 phenotype. These include arginase metabolism, fatty acid synthesis, and expression of the vascular adhesion molecule CD84. These phenomena are associated with peripheral monocyte phenotypic conversion and trafficking into diseased tissues. These activities for BRI3 are consistent with the elevation of BRI3 specifically in the CD16 subset that we observed.

Based on the foregoing, inhibiting BRI3 activity should provide a novel means for preventing the development of monocytes of the CD16 or M2 phenotype and/or for preventing/inhibiting the trafficking/migration of monocytes into inflamed sites such as in the central nervous system and for preventing/inhibiting monocyte-associated neuroinflammation by preventing activated monocytes from regulating aspects of the M2 phenotype and from entering damaged tissues including the brain.

It is known that the actions of microglia, the resident myeloid cells of the CNS parenchyma, may diverge from, or intersect with, those of recruited monocytes to drive immune-mediated pathology. However, defining the precise roles of monocytes to neuroinflammation has historically been impeded by the lack of discriminating markers and experimental systems capable of accurately identifying them. The present invention addresses this need.

The discovery that monocyte trafficking to the CNS can be prevented or inhibited by blocking/inhibiting BRI3 expression and/or activity is of great potential clinical significance since inflammation and in particular neuroinflammation generally increases with aging and is a well-characterized component of age-related and genetic neurological disorders. Moreover, monocytes circulating in blood can be activated by signals from injured or degenerating tissues, as well as environmental toxicants, virus, and traumatic injury. Once activated, certain monocytes are recruited to tissues where they can assume a non-classical M2 phenotype. M2 monocytes may initially have positive impacts such as phagocytosis of damaged cells and suppression of tissue inflammation (Understanding the Mysterious M2 Macrophage through Activation Markers and Effector Mechanisms. Rőszer T. Mediators Inflamm. 2015; 2015:816460. doi: 10.1155/2015/816460. Epub 2015 May 18.PMID: 26089604). Unfortunately, extensive infiltration of monocytes into the brain can damage tissues and exacerbate neuroinflammatory conditions. This is a major problem in chronic diseases of the elderly such as Alzheimer's and Parkinson's, as well as auto-immune disorders like multiple sclerosis, damage caused by intoxication or head injury, and cerebral infarction.

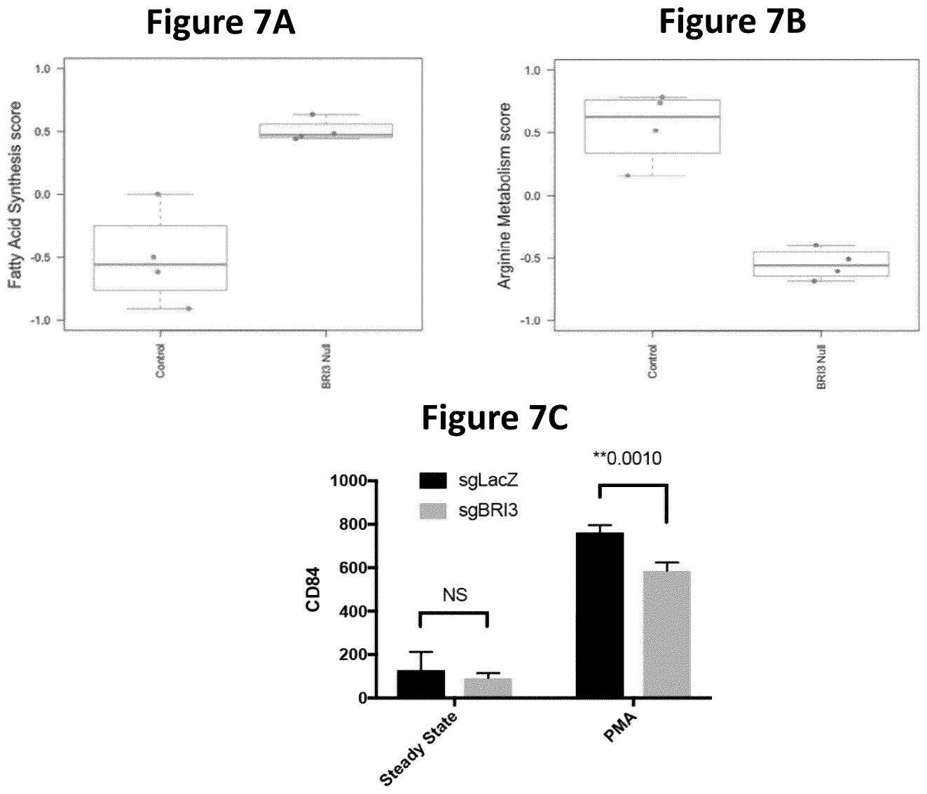

Arginine metabolism is a primary marker of the M2 phenotype. Recent studies indicate the potential for deleterious effects associated with the presence M2 monocytes, associating arginase expression with neurological disorders and brain injury (“Arginase: A Multifaceted Enzyme Important in Health and Disease”, R William Caldwell 1, Paulo C Rodriguez 1, Haroldo A Toque 1, S Priya Narayanan 1, Ruth B Caldwell 1 PMID: 29412048, PMCID: PMC5966718, DOI: 10.1152/physrev.00037.2016). As disclosed infra, we have shown that that inactivation of BRI3 impacts gene expression related to critical molecular checkpoints involved in the transition for the M2 phenotype. Under differentiation conditions, BRI3 loss led to reproducible dysregulation in genes associated with fatty acid synthesis and arginine metabolism compared with control cells (FIG. 7A, B). This finding was also associated with reductions in the expression of the CD84 vascular adhesion molecule (FIG. 7C). Arginine metabolism and fatty acid synthesis are a key features of infiltrating monocytes and phagocytic transition but mechanisms underlying these phenotypes poorly understood.

Our discoveries indicate that BRI3, a gene we found to be highly elevated in CD16 monocytes in Parkinson's, likely modulates the M2 phenotypic transition of monocytes. Based on our BRI3 inactivation data, we predict that the elevated BRI3 we identify in Parkinson's, and likely other inflammatory and particularly other neuroinflammatory conditions, is participating in the peripheral monocytes response to damaged brain tissue, namely the assumption of the infiltrative M2 phenotype. These observations extend the scope of our previous discoveries beyond the modification of inflammatory phenotypes by providing a framework to modulate monocyte trafficking and the conversion of monocytes to the M2 phenotype.

This is critically important because it allows for the development of methods and agents to target cells in the periphery before they enter the brain, further propagating neuroinflammation and shielded from many therapies by the blood brain barrier. Beyond sterile inflammatory disorders, it is also noteworthy that significant immune cell invasion into the brain has been implicated in the neurological sequelae associated with acute viral infection, most recently SARS-COV-2. Our discoveries are novel because we have identified a constellation of signaling events associated with BRI3, providing avenues to target the poorly characterized M2 transition, monocyte trafficking, and related energy metabolism in a panoply of neurological disorders.

The advantage is that we have identified targets related to a virtually uncharacterized molecular pathway linked to PD and likely other inflammatory conditions wherein disease pathology similarly involves the infiltration and adverse effects associated with M2 monocytes, and wherein such pathway is likely representative of a broadly acting immune response to neurological damage and stress. Monocytes are an exciting target because they represent a first responder population that can sense a wide range of peripheral insults and tissue damage. Importantly, they enter the brain and cause problems. Their role is likely under-recognized because in reality once they infiltrate the brain in their activated state they are virtually indistinguishable from microglia. Another key upside is that they can be sampled using a simple blood draw.

As earlier noted, it is known that during some insults to the brain, circulating monocytes can be mobilized to breach the BBB, migrate into the brain, and subsequently contribute to the neuroimmune response in association with microglia (Prinz, et al., “Heterogeneity of CNS myeloid cells and their roles in neurodegeneration”, Nat Neurosci 14, 1227-1235 (2011), Mildner, et al., CCR2+Ly-6Chi monocytes are crucial for the effector phase of autoimmunity in the central nervous system. Brain 132, 2487-2500 (2009)). Peripheral monocytes are known to enter the brain after traumatic brain injury and contribute to neuronal injury (S Gyoneva, et al., Ccr2 deletion dissociates cavity size and tau pathology after mild traumatic brain injury. J Neuroinflammation 12, 228 (2015), and they play a similar role in multiple sclerosis mouse models (Fife et al., “CC chemokine receptor 2 is critical for induction of experimental autoimmune encephalomyelitis”, J Exp Med 192, 899-905 (2000); Izikson et al., “Resistance to experimental autoimmune encephalomyelitis in mice lacking the CC chemokine receptor (CCR) 2”, J Exp Med 192, 1075-1080 (2000). In the experimental autoimmune encephalomyelitis (EAE) rodent model of multiple sclerosis, gene-expression profiles indicate that infiltrating monocytes are highly inflammatory compared with microglia. Moreover, invading monocytes induce axonal damage by initiating demyelination, whereas microglia clear debris (R Yamasaki, et al., Differential roles of microglia and monocytes in the inflamed central nervous system. J Exp Med 211, 1533-1549 (2014)).

Also, as further previously noted it is well-described in models of trauma, neurological disease, or infection that inflammatory BM-derived monocytes traffic to and are recruited into inflamed tissue, including the brain and spinal cord (Donnelly, D. J., and Popovich, P. G. (2008). “Inflammation and its role in neuroprotection, axonal regeneration and functional recovery after spinal cord injury”, Exp. Neurol. 209, 378-388; Kigerl, et al., (2009), “Identification of two distinct macrophage subsets with divergent effects causing either neurotoxicity or regeneration in the injured mouse spinal cord”, J. Neurosci. 29, 13435-1344; McGavern et al., (2011), “Illuminating viral infections in the nervous system”, Nat. Rev. Immunol. 11, 318-329). Recent evidence also indicates that there is significant trafficking and recruitment of peripherally derived monocytes to the brain with psychological stress (Brevet et al. (2010), “Chronic foot-shock stress potentiates the influx of bone marrow-derived microglia into hippocampus”, J. Neurosci. Res. 88, 1890-1897; Wohleb, et al., (2013), “Stress-induced recruitment of bone marrow-derived monocytes to the brain promotes anxiety-like behavior”, J. Neurosci. 33, 13820-13833; Ataka et al. (2013), “Bone marrow-derived microglia infiltrate into the paraventricular nucleus of chronic psychological stress-loaded mice”, PLOS ONE 8: e81744; Wohleb et al., (2014b), “Knockdown of interleukin-1 receptor type-1 on endothelial cells attenuated stress-induced neuroinflammation and prevented anxiety-like behavior”, J. Neurosci. 34, 2583-2591; Sawada et al., (2014) “Suppression of bone marrow-derived microglia in the amygdala improves anxiety-like behavior induced by chronic partial sciatic nerve ligation in mice”, Pain 155, 1762-1772). In these studies, monocytes traffic to the brain and differentiate into brain macrophages that promote inflammatory signaling.

Thus, based on the foregoing, and our observations, inhibiting the formation of M2 monocytes and/or inhibiting trafficking of BM-derived monocytes to an inflamed site such as in the CNS or brain by inhibiting BRI3 activity and/or expression represents a viable means for treating such neuroinflammatory conditions. In particular, inhibiting the formation of M2 monocytes and/or inhibiting trafficking of BM-derived monocytes to inflamed sites such as in the CNS or brain by inhibiting BRI3 activity and/or expression is useful in treating and/or preventing and/or delaying the onset of neurodegenerative diseases such as Parkinson's disease (PD), and other neuroinflammatory conditions previously identified.

In particular inhibiting the formation of M2 monocytes and/or inhibiting trafficking of BM-derived monocytes to an inflamed site such as in the CNS or brain by inhibiting BRI3 activity and/or expression represents a viable means for treating acute or chronic inflammatory conditions caused by an aging, infection, an environmental insult, an injury such as a repetitive motion injury, or an autoimmune or inflammatory condition.

Also inhibiting the formation of M2 monocytes and/or inhibiting trafficking of BM-derived monocytes to an inflamed site such as in the CNS or brain by inhibiting BRI3 activity and/or expression represents a viable means for treating/preventing an acute or chronic neuroinflammatory condition caused by aging, an infection, an environmental insult, an injury such as a traumatic brain injury, an autoimmune condition, a sterile inflammatory condition, a demyelinating condition, stress, stroke or a neurodegenerative disease or other inflammatory condition that affects the central nervous system (CNS).

More specifically, inhibiting the formation of M2 monocytes and/or inhibiting trafficking of BM-derived monocytes to an inflamed site such as in the CNS or brain by inhibiting BRI3 activity and/or expression represents a viable means for treating a subject who is at risk of developing an inflammatory condition because of heredity, environmental exposure or an activity such as a sports activity such as football, boxing, wrestling, combat correlated to a neuroinflammatory condition.

More specifically, inhibiting the formation of M2 monocytes and/or inhibiting trafficking of BM-derived monocytes to an inflamed site such as in the CNS or brain by inhibiting BRI3 activity and/or expression represents a viable means for treating inflammation or neuroinflammation caused by an infectious agent, e.g., a virus, prion, bacteria, parasite, yeast or fungus. Specific examples include inflammatory conditions caused by a coronavirus (e.g., SARS-COVID or SARS COVID 2 (Covid 19)), Japanese encephalitis virus, influenza virus, measles virus, herpes simplex virus, varicella zoster virus, mumps virus, chicken pox virus, rubella virus, Streptococcus pneumoniae, Mycoplasma pneumoniae, human metapneumovirus (HPMV), human parainfluenza virus, Legionnaires' disease, respiratory syncytial virus (RSV), rhinovirus, pneumocytitis pneumonia, pneumococcal disease, et al.

Further, inhibiting the formation of M2 monocytes and/or inhibiting trafficking of BM-derived monocytes to an inflamed site such as in the CNS or brain by inhibiting BRI3 activity and/or expression represents a viable means for treating neurodegenerative diseases such as Alzheimer's disease, Parkinson's disease (PD), senility or another memory disorder, ataxia, motor neuron disease, multiple sclerosis (MS), Lewy body disease or Lewy body dementia (LBD), multiple system atrophy (MSA), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)), optionally wherein the neurodegenerative diseases is PD.

Also, inhibiting the formation of M2 monocytes and/or inhibiting trafficking of BM-derived monocytes to an inflamed site such as in the CNS or brain by inhibiting BRI3 activity and/or expression represents a viable means for treating demyelinating conditions such as multiple sclerosis, ALS, Transverse Myelitis, Guillain-Barré syndrome, Neuromyelitis optica, or Devic's disease, an Idiopathic inflammatory demyelinating disease, a Leukodystrophic or dysmyelinating disorder, Central pontine myelinolysis, a myelopathy such as tabes dorsalis (syphilitic myelopathy), Leukoencephalopathy such as progressive multifocal leukoencephalopathy, a Leukodystrophy, chronic inflammatory demyelinating polyneuropathy, anti-MAG peripheral neuropathy, Charcot-Marie-Tooth disease, Hereditary neuropathy and Progressive inflammatory neuropathy.

Yet further, inhibiting the formation of M2 monocytes and/or inhibiting trafficking of BM-derived monocytes to an inflamed site such as in the CNS or brain by inhibiting BRI3 activity and/or expression represents a viable means for treating autoimmune diseases that affect the CNS such as Neuromyelitis optica, Anti-myelin oligodendrocyte glycoprotein antibody disease (MOG), Acute disseminated encephalomyelitis (ADEM), Chronic meningitis, Central nervous system (CNS) vasculitis, Hashimoto's encephalitis, Steroid responsive encephalopathy associated with autoimmune thyroiditis (SREAT), Neurosarcoidosis, Optic neuritis or Transverse myelitis.

Additionally, inhibiting the formation of M2 monocytes and/or inhibiting trafficking of BM-derived monocytes to an inflamed site such as in the CNS or brain by inhibiting BRI3 activity and/or expression represents a viable means for treating chronic or acute inflammation, e.g., neuroinflammation or myocarditis caused by a vaccine.

Also, inhibiting the formation of M2 monocytes and/or inhibiting trafficking of BM-derived monocytes to an inflamed site such as in the CNS or brain by inhibiting BRI3 activity and/or expression represents a viable means for treating chronic or acute inflammation is caused by concussive injuries such as caused by a sports activity or by stroke or sepsis or acute respiratory disease syndrome (ARDS).

In some embodiments such treatment or prophylaxis may include the administration or use of one or more other agents or drugs or other treatments useful for treatment or prophylaxis of the neurodegenerative disease or neuroinflammation, e.g., one or more other agents or treatments useful for treatment or prophylaxis of PD.

These other agents or treatments may be administered separately or in combination with the inventive methods of treatment or prevention. In particular such agents or drugs or other treatments may include any drug used to treat inflammation or neuroinflammatory conditions, e.g., one or more of the following:

(i) levodopa alone or in combination with carbidopa and benserazide (which are dopa decarboxylase inhibitors that do not cross the blood-brain barrier and inhibit the conversion of levodopa to dopamine outside the brain, reducing side effects and improving the availability of levodopa for passage into the brain). These drugs optionally may comprise by way of example controlled-release (CR) versions of levodopa or extended-release levodopa formulations, oral, longer-acting formulations, as well as inhaled or transdermal formulations.
(ii) COMT or COMT inhibitors Catechol-O-methyltransferase (COMT) optionally may be used in conjunction with levodopa/carbidopa when a person is experiencing the “wearing off phenomenon” with their motor symptoms. COMT inhibitors which may be used to treat PD and end-of-dose motor fluctuations include by way of example opicapone, entacapone, and tolcapone.
(iii) Other Dopamine agonists such as
pergolide, pramipexole, ropinirole, piribedil, cabergoline, apomorphine, bromocriptine, and lisuride.
(iii) MAO-Inhibitors such as safinamide, selegiline and rasagiline which increase the amount of dopamine in the basal ganglia by inhibiting the activity of monoamine oxidase B, an enzyme that breaks down dopamine.
(v) Other drugs such as amantadine and anticholinergics such as quetiapine or pimavanserin for psychosis, cholinesterase inhibitors for dementia, and modafinil, doxepin and rasagline for excessive daytime sleepiness.
(v) Combination drugs such as Nourianz (istradefylline), a recently approved add-on treatment to levodopa/carbidopa in adult patients with Parkinson's disease (PD) experiencing “off” episodes.
(vi) Other Treatments (non-drug treatments) such as other gene therapies, cell-based therapies, and surgery. Surgery for PD includes lesional and deep brain stimulation (DBS). Target areas for DBS or lesions include the thalamus, globus pallidus, or subthalamic nucleus. DBS involves the implantation of a medical device called a neurostimulator, which sends electrical impulses to specific parts of the brain. Other, less common surgical therapies involve intentional formation of lesions to suppress overactivity of specific subcortical areas. For example, pallidotomy involves surgical destruction of the globus pallidus to control dyskinesia.
Four areas of the brain have been treated with neural stimulators in PD, i.e., the globus pallidus interna, thalamus, subthalamic nucleus, and pedunculopontine nucleus. DBS of the globus pallidus interna improves motor function, while DBS of the thalamic DBS improves tremor.
Examples are provided below to illustrate the present invention. These examples are not meant to constrain the present invention to any particular application or theory of operation.

### EXAMPLES

### Example 1: Identification of Genes Wherein Expression is Modified in PD Patients

Peripheral immune cells are executors of the immune inflammatory response. Based thereon we hypothesized that if innate immune inflammatory responses correspond with the brain pathology in PD or neural inflammation, peripheral immune cells and/or peripheral immune responses potentially represent an easily sampled surrogate for detection of neural inflammation occurring in association with PD and potentially other neural diseases associated with neural inflammation.

A growing number of studies implicate monocyte dysregulation in PD, but there is still not enough known about the PD-associated monocyte state. We leveraged newly available single-cell transcriptomic technologies to better characterize monocytes in PD in order to identify gene expression signatures and pathways of interest for the management of the disease. Using this approach, we integrated and analyzed over 17,000 peripheral blood mononuclear cells (PBMCs) obtained from a small cohort of PD patients and controls. Deep sequencing at single cell resolution characterizing monocytes revealed panels of genes differentially expressed in subsets of PD monocytes compared with controls. We validated BRI3, a gene consistently upregulated in the CD16 subset of PD monocytes, using biochemical techniques. As shown herein we identified elevated BRI3 protein expression in activated cultured human monocytes and in the extracellular vesicle fraction of PD patient plasma. We inactivated BRI3 in the THP1 monocyte cell line and demonstrate that BRI3 loss reduces inflammatory cytokine secretion in cultured monocytes. Taken together the study supports the existence of a PD-specific monocyte cell state and identifies BRI3 as a novel indicator, and likely regulator, of innate immune perturbation in PD.

### Methods

Rapidly emerging technology now allows for RNA-sequencing at single-cell resolution (scRNA-Seq) (Hwang, B., J. H. Lee, and D. Bang, Single-cell RNA sequencing technologies and bioinformatics pipelines. Exp Mol Med, 2018. 50 (8): p. 96). Data analysis protocols are continuously evolving with first-generation studies focused on single or paired sample comparisons identifying new cellular subtypes (Ximerakis, M., et al., Single-cell transcriptomic profiling of the aging mouse brain. Nat Neurosci, 2019. 22 (10): p. 1696-1708). and transcriptomic states (Reitman, Z. J., et al., Mitogenic and progenitor gene programmes in single pilocytic astrocytoma cells. Nat Commun, 2019. 10 (1): p. 3731). We also identified a small cohort of patients suitable for analysis as an integrated dataset in order to make determinations regarding the state of peripheral immune cells in PD. This cohort was limited to male patients for three reasons: (1) in order to remove variation due to sex chromosome dosage; (2) to circumvent the now recognized confound of heterogeneous X inactivation across individual female cells (Garieri, M., et al., Extensive cellular heterogeneity of X inactivation revealed by single-cell allele-specific expression in human fibroblasts. Proc Natl Acad Sci USA, 2018. 115 (51): p. 13015-13020); and (3) We were able to identify 2 healthy controls and 3 PD male volunteers of similar age whose medical history did not indicate co-morbidities likely to influence the innate immune system in a reasonable timeframe (Table 1). PD volunteers represented a spectrum of PD progression with time since diagnosis ranging from 1 to 6 years and UPDRS scores from 8 to 56. Patients varied in their clinical medication regimes that included 1 patient using oral levodopa/carbidopa, 1 prescribed ropinirole and rasagiline, and one early-stage participant taking part in a clinical study involving a cAbl inhibitor. Patients all self-report use of over the counter anti-inflammatory medications of various brands.

TABLE 1

| | Controls (2 males) | PD (3 males) |
|---|---|---|
| Ages (years) | 60.5 (0.7) | 71.4 (13.2) |
| Ethnicity (% non-hispanic) | 100 | 100 |
| Disease duration (years) | N/A | 3.5 (2.0) |
| UPDRS (total) | N/A | 29.7 (24.3) |
| Dopamine replacement (%) | N/A | 33.3 (57.7) |
| Dopamine agonist (%) | N/A | 33.3 (57.7) |
| Dopamine anti-degradation (%) | N/A | 33.3 (57.7) |

Values are depicted as mean (SD) unless otherwise noted.
Dopamine replacement: carbidopa/levodopa; dopamine agonist; ropinirole, pramipexole; dopamine anti-degradation; rasagiline, entacapone

Clinical heterogeneity is observed in PD patients as the result of aging, lifestyle, and environmental factors, among others. A key advance made by authors is the creation of an original computational pipeline, drawing together multiple programming strategies to integrate the data and allow for the interrogation of changes in gene expression in individual cell types. Preprocessing and quantification of gene expression levels was performed using CellRanger v3.1. Briefly, reads associated with each sample were aligned by indexing to GRCh38 (GENCODE v.24) using STAR v.2.5.0, trimming read 2 to remove 3′ poly(A) tails (>7 A's) and discarding fragments with fewer than 24 remaining nucleotides as described (Yuan, J., et al., “Single-cell transcriptome analysis of lineage diversity in high-grade glioma”, Genome Med, 2018. 10 (1): p. 57). Aligned reads were then assigned to cell-specific barcodes and PCR duplicates removed by counting Unique Molecular Identifier (UMI) sequences. 1 base-pair error correction will be applied to both UMIs and cell-barcodes to account for sequencing errors. Per-gene UMI counts will be used to generate a preliminary gene expression matrix. Subsequently, outputs from CellRanger were loaded into the R statistical programming environment (v 3.6.1) using the Seurat v3 R package (Stuart, T., et al., “Comprehensive Integration of Single-Cell Data”, Cell, 2019. 177 (7): p. 1888-1902 e21) and subjected to additional quality control procedures. Cells exhibiting high proportions of reads mapping to the mitochondrial genome or low expression complexity (based on total UMI count and number of detected features) were removed from downstream analysis (Szabo, P. A., et al., “Single-cell transcriptomics of human T cells reveals tissue and activation signatures in health and disease”, Nat Commun, 2019. 10 (1): p. 4706). Expression of key cell-cycle regulators determined potential variation across the dataset attributable to cell-cycle phase. If determined to explain a significant proportion of gene expression differences, variability attributable to cell cycle stage was statistically regressed out during the normalization step. Normalization of raw counts was performed using the recently described SCTransform method implemented in Seurat v3. Finally, corresponding gene expression profiles shared across cells from independent samples will be identified using recently developed canonical correlation analysis (CCA)-based method implemented in Seurat v3, and used to perform dataset integration and batch correction, ultimately producing a single dataset to be used for all downstream analyses (Hafemeister, C. and R. Satija, Normalization and variance stabilization of single-cell RNA-seq data using regularized negative binomial regression. Genome Biol, 2019. 20 (1): p. 296).

### Results

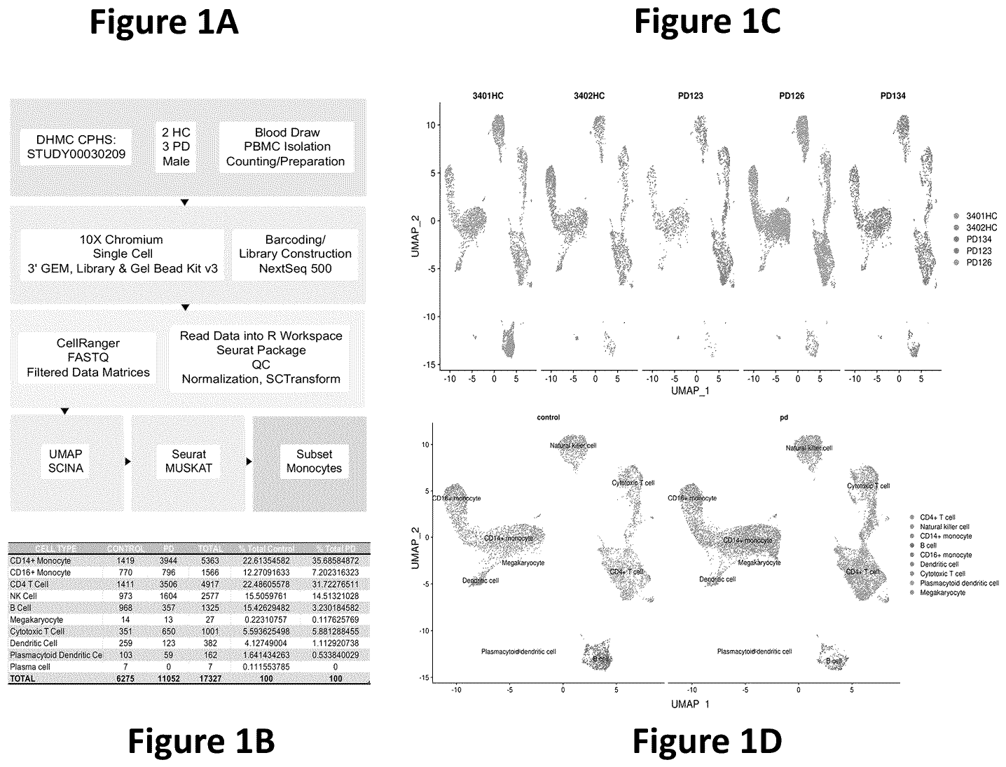

High variability is a concern in the analysis of transcriptomic state in highly reactive human cell populations in aging individuals diagnosed with complex and slowly progressive diseases like PD and treated using different therapeutic regimes. We collected whole blood from five volunteers (n=2 healthy control, 3 PD) using clinical methodologies consistent with the widely-accepted protocols laid out by the Parkinson's Progression Marker's Initiative (“The Parkinson Progression Marker Initiative (PPMI)”, Prog Neurobiol, 2011. 95 (4): p. 629-35) making minor changes in the blood draw procedure for optimal PMBC isolation. The overall workflow is presented in FIG. 1A. Single cell suspension containing purified PBMCs were processed with Chromium Single Cell B Chip (10× Genomics) and pooled libraries were sequenced using the NextSeq500 instrument. Preprocessed data from experimental cells selected as described above was subject to dimensionality reduction using uniform manifold approximation and projection (UMAP) (Becht, E., et al., “Dimensionality reduction for visualizing single-cell data using UMAP”, Nat Biotechnol, 2018) to aggregate cells of similar transcriptional profiles. Resulting cell clusters were assigned cellular identities using the prior knowledge-based SCINA algorithm (Zhang, Z., et al., “SCINA: A Semi-Supervised Subtyping Algorithm of Single Cells and Bulk Samples”, Genes (Basel), 2019. 10 (7)). As expected, we collected more cells from the larger PD group, but this discrepancy did not influence cell clustering during data integration (FIGS. 1B and C). We achieved reasonable segregation of the major subcategories anticipated in PBMC populations (FIG. 1D). No cluster was driven by over-representation of cells from a single individual (FIGS. 1C and D). Close inspection of the data however indicated changes in the proportions of cell populations in PD compared with health controls. Despite analyzing more cells overall, in PD patients we identified relatively fewer CD16 monocytes, B cells, and dendritic cells compared with controls. In contrast, we detected increased numbers of CD14 monocytes and CD4 T cells in PD patients compared with controls (FIGS. 1B and D). The finding of changes in the CD14/CD16 ratio in PD is consistent with previous reports analyzing immune cells in larger cohorts indicating increased CD14 monocytes in PD and decreases in the CD16 subset (Grozdanov, V., et al., “Inflammatory dysregulation of blood monocytes in Parkinson's disease patients”, Acta Neuropathol, 2014. 128 (5): p. 651-63). Data indicate that the anticipated cell populations were identified and sufficiently represented for each patient the integrated dataset despite the clinical and sampling variability.

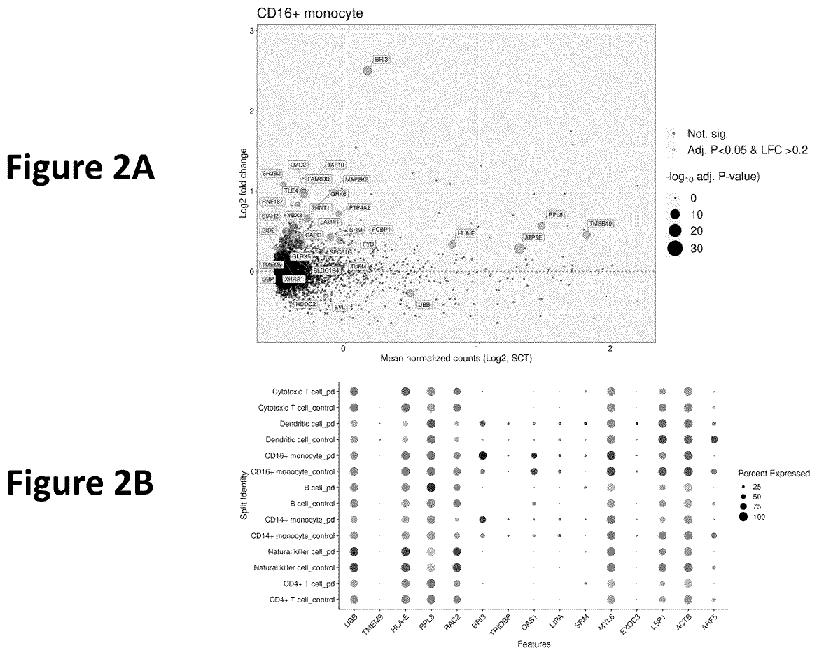

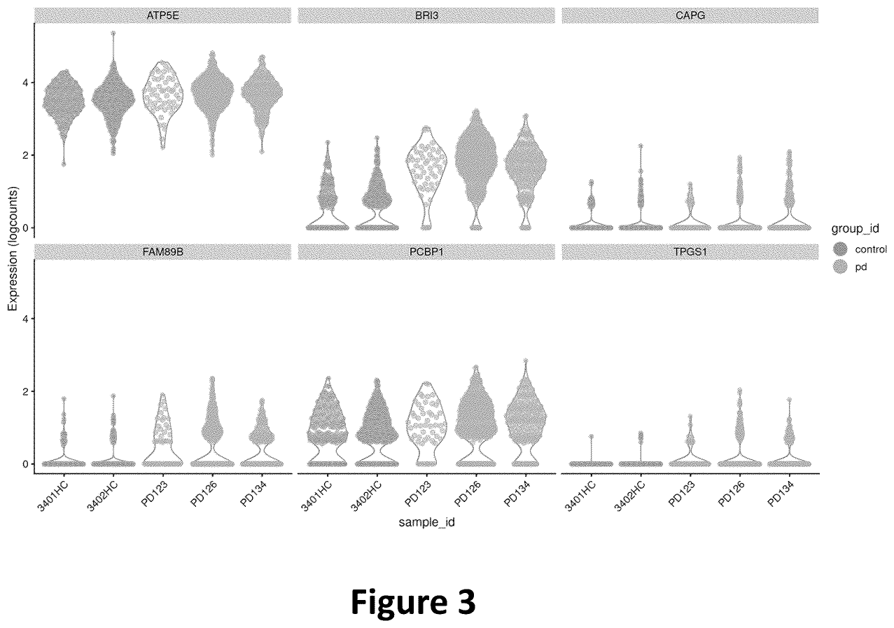

Methodologies for analysis of differential gene expression in scRNA-Seq datasets continue to evolve. Challenges arising based on the sparsity of the data are augmented by the clinical complexity of diseases like PD, variation in the number of samples available per group, number of cells and cell subpopulations identified in individuals, and the magnitude of differential expression. To overcome these challenges we used a recently described analysis pipeline that accounts for cell and sample-level changes, utilizing mixed-modeling strategies in data normalized by an internal anchored reference dataset (muscat R package, (Helena L. Crowell, C. S., Pierre-Luc Germain, Daniela Calini, Ludovic Collin, Catarina Raposo, Dheeraj Malhotra, Mark D. Robinson, “On the discovery of subpopulation-specific state transitions from multi-sample multi-condition single-cell RNA sequencing data”, bioRxiv, 2020)). Among genes observed to be significantly altered in PD, we identified several genes of interest that withstood this rigorous statistical analysis in the CD16 subset (FIG. 2A-B). The mixed modeling analysis strategy we employed was designed expressly to normalize multi-sample, multi-group, multi-(cell-) subpopulation scRNA-seq data (Helena L. Crowell, C. S., Pierre-Luc Germain, Daniela Calini, Ludovic Collin, Catarina Raposo, Dheeraj Malhotra, Mark D. Robinson, “On the discovery of subpopulation-specific state transitions from multi-sample multi-condition single-cell RNA sequencing data”, bioRxiv, 2020); however, previous reports highlight substantial variability in monocyte gene expression in PD (Schlachetzki, J. C. M., et al., “A monocyte gene expression signature in the early clinical course of Parkinson's disease”, Sci Rep, 2018. 8 (1): p. 10757). To further confirm differentially expressed genes in our cohort, we evaluated genes of the highest significance individually across all 5 individuals. Analysis of gene expression in the five individuals highlighted variability in the total number of cells detected with high levels of expression of the genes identified as differentially expressed using the mixed modeling strategy. We evaluated CD16 monocytes and found changes that were more robust than we had observed analyzing CD14 monocytes. Increased expression of ATP5E, BRI3, PCBP, and FAM89B (encoding ATP synthase F1 subunit epsilon, brain protein 13, poly (rC) binding protein 1, and family with sequence similarity 89 member B, respectively) were consistently observable in PD patients compared with controls (FIG. 3). These findings indicate several genes whose expression is convincingly altered in PD compared with controls. Data indicate robust alterations in gene expression in the CD16 non-classical or “patrolling” monocyte subpopulation, typically associated with the modulation of inflammatory processes, maintenance of vascular endothelial homeostasis, responding to tissue injury and chronic inflammatory disease (Narasimhan, P. B., et al., “Nonclassical Monocytes in Health and Disease”, Annu Rev Immunol, 2019. 37: p. 439-456). The findings support previous reports indicating detectable alterations in the state of PD monocytes (Grozdanov, V., et al., “Inflammatory dysregulation of blood monocytes in Parkinson's disease patients”, Acta Neuropathol, 2014. 128 (5): p. 651-63; Schlachetzki, J. C. M., et al., “A monocyte gene expression signature in the early clinical course of Parkinson's disease”, Sci Rep, 2018. 8 (1): p. 10757; and Nissen, S. K., et al., “Alterations in Blood Monocyte Functions in Parkinson's Disease”, Mov Disord, 2019. 34 (11): p. 1711-1721), and extend these studies using high-resolution transcriptomics to identify new genes of interest for delineation of the complexities of monocyte dysregulation in PD.

As shown herein we have discovered several genes elevated in PD across multiple cell types. Among them, we focused on the robust and reproducible enrichment of BRI3 transcript in a large number of CD16 monocytes PD patients (FIG. 3). BRI3 reportedly is expressed on some hematopoietic cells and is in a list of genes identified using screening strategies to define changes related to hematopoietic maturation (Mello, F. V., et al., “Maturation-associated gene expression profiles along normal human bone marrow monopoiesis”, Br J Haematol, 2017. 176 (3): p. 464-474). Mello also cites two earlier publications tangentially relating BRI3 biology to the cytokine response. In the first article published in 2003, the authors reportedly observed that the suppression of BRI3 expression resulted in resistance to tumor necrosis factor-induced cell death in cultured fibroblasts (Wu et al., “bri3, a novel gene, participates in tumor necrosis factor-α-induced cell death”, Biochem Res. Comm. 311 (2) pp. 528-34). In a second publication, published in 2018, the authors allege that BRI3 may interact with IFITM3, a modulator of the interferon response, in a yeast to-hybrid assay (Akiva et al., “Identification of IFITM3 and MGAT1 as novel interaction partners of BRI3 by yeast two-hybrid screening”, Turk J Biol., 42 (6): 463-470).

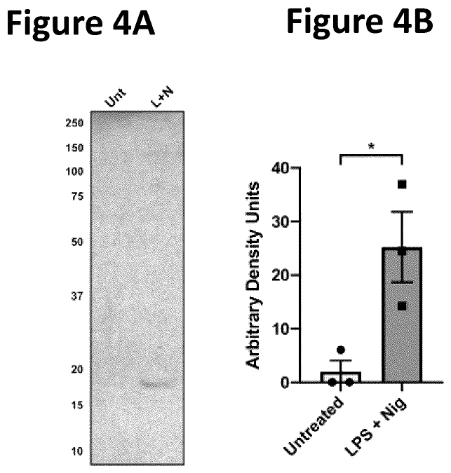

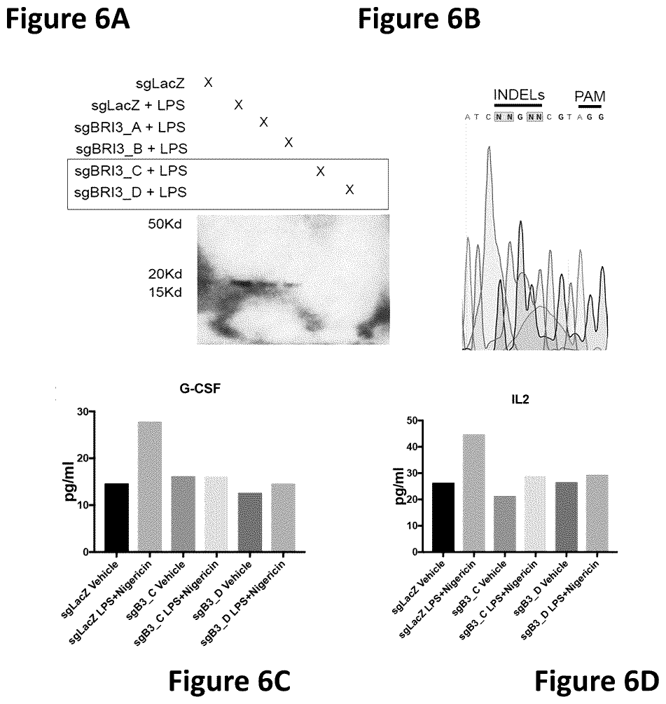

By contrast, our results suggest that BRI3 has an immune modulating function in PD innate immune cells, especially in monocytes and closely-related CNS microglia. Our supposition that BRI3 functions in innate immune cells like monocytes is supported by our biological validation of scRNA-Seq studies in which we have observed that BRI3 was inducible in cultured human monocytes exposed to inflammatory insult (cultured in the presence of LPS and nigericin) (FIG. 4A-B), induced under standardized differentiation conditions promoting the the M2/non-classical/pro-repair phenotype (J Immunol Methods, 2020 March; 478:112721. doi: 10.1016/j.jim.2019.112721. Epub 2020 Feb. 4), and that BRI3 inactivation using CRISPR/CAS9 reduced the cytokine response in the same monocyte cell line (FIG. 6A-D).

### Example 2: Diagnosis of PD

Currently there is no definitive test for diagnosing PD. Rather, diagnosis is generally made based on symptoms, such as tremor, impaired movements and balance, changes in speech and writing, tests that rule out other neurological diseases, and response to carbidopa-levodopa.

According to the present discovery, PD or an increased risk for developing PD may be diagnosed based on the aberrant (increased) expression of BRI3 and, in certain examples, other genes related to the monocyte cell state. Subjects suspected to have PD or those having an increased risk for developing PD, e.g., because of a family history or environmental factors (e.g., prolonged exposure to pesticides and herbicides; Vietnam-era exposure to Agent Orange; prolonged exposure to heavy metals, detergents and solvents) will be tested. For example, subjects who show at least one symptom that may be of PD and/or subjects at risk for developing PD (e.g., genetically predisposed subject) will be tested. BRI3 elevation or related changes in the monocyte state may indicate PD, risk of developing PD, occupation, lifestyle, or environmental exposure that increase risk of developing PD, or a clinical condition or lifestyle with the potential to accelerate the progression of PD.

Peripheral blood samples will be collected from the subjects and peripheral blood mononuclear cells (PBMCs) will be isolated using a standard protocol. Additionally, or alternatively, cerebrospinal fluid (CSF) will be collected from the subject and cell contents will be isolated using a standard protocol.

### Transcript Analyses

In one test, transcript analyses on the PBMCs and/or CSF cells will be performed. For example, single cells will be first obtained and single-cell RNA sequencing, single-cell quantitative PCR (qPCR), or single-cell nanostring may be used. Alternatively, cells may be presorted into different cell types (e.g., immune cell types) and then gene expression may be determined via quantitative PCR (qPCR).

Transcripts of genes such as BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5 in various cell types may be analyzed. Alternatively, at least the transcript levels of BRI3 may be analyzed. If BRI3 expression is significantly higher than that of a healthy control or than a standard level, for example in monocytes (e.g., CD16⁺ monocytes and/or CD14⁺ monocytes), the subject will be diagnosed as having PD.

Transcript levels of other genes measured may also be taken into account towards diagnosis. For example, upregulation of one or more of FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM and/or downregulation of one or more of HDDC2, EVL, UBB, RAC2, OAS1, LSP1, or ARF5 may be further included as a criteria for diagnosing PD. For instance, downregulation of LSP1 in cytotoxic T cells (or CD8⁺ T cells), CD16⁺ monocytes, CD14⁺ monocytes, and/or NK cells, and/or downregulation in ARF5 in cytotoxic T cells (or CD8⁺ T cells), dendritic cells, CD16⁺ monocytes, B cells, CD14⁺ monocytes, NK cells, and/or CD4⁺ T cells may be further included as a criteria for diagnosing PD. Changes in the population of immune cells may also be taken into account towards diagnosis. For example, reduction in CD16⁺ monocytes, B cells, and/or dendritic cells and/or increase in CD14⁺ monocytes and/or CD4⁺ T cells may be further included as a criteria for diagnosing PD.

### Protein Expression Analyses

In another test, PBMCs and/or CSF cells be stained, e.g., with antibodies, for various immune cell markers (e.g., one or more of CD45, CD45RO, CD45RA, CD1a, CD3, CD4, CD8, CD11b, CD11c, CD14, CD16, CD19, CD20, CD34, CD40, CD56, CD64, CD68, CD71, CD80, CD83, CD86, CD94, CCR5, FceRIa, HLA-DR) and for BRI3, and the protein expression levels will be determined by, e.g., flowcytometry. If BRI3 expression is significantly higher than that of a healthy control or than a standard level, for example in monocytes (e.g., CD16⁺ monocytes and/or CD14⁺ monocytes) and/or dendritic cells, the subject will be diagnosed as having PD.

Changes in the expression of other genes may also be taken into account towards diagnosis. For example, downregulation of LSP1 in cytotoxic T cells (or CD8⁺ T cells), CD16⁺ monocytes, CD14⁺ monocytes, and/or NK cells, and/or downregulation in ARF5 in cytotoxic T cells (or CD8⁺ T cells), dendritic cells, CD16⁺ monocytes, B cells, CD14⁺ monocytes, NK cells, and/or CD4⁺ T cells may be further included as a criteria for diagnosing PD. Changes in the population of immune cells may also be taken into account towards diagnosis. For example, reduction in CD16⁺ monocytes, B cells, and/or dendritic cells and/or increase in CD14⁺ monocytes and/or CD4⁺ T cells may be further included as a criteria for diagnosing PD.

### Extracellular-Released Vesicle Analysis

Extracellular-released vesicles (EVs), such as exosomes and microvesicles (MVs), are released from cell membranes and contain various biological materials such as nucleic acids (e.g., RNA) and proteins of the cell of origin. Recently, neuronal exosomes are shown to be a great tool for detecting important disease information in a subject (Jiang C. et al., J Neurol Neurosurg Psychiatry, 2020 July; 91 (7): 720-729. doi: 10.1136/jnnp-2019-322588. Epub 2020 Apr. 9). According to the present discovery, microglia express high levels of BRI3 in PD patients, and therefore PD or an increased risk of developing PD will be diagnosed based on an increased level of BRI3 in the EVs (exosomes and/or MVs).

Blood samples and/or CSF samples will be obtained from subjects. Exosomes will be isolated using a standard protocol (e.g. ultracentrifugation and size exclusion chromatography) and lysed. Exosomal RNA and proteins will be extracted using a standard protocol. BRI3-encoding RNA and/or BRI3 protein will be quantified using a standard method (e.g., qPCR for RNA and enzyme-linked immunosorbent assay (ELISA) for protein).

### Neuroimaging

In current diagnosis of PD, imagining tests such as an MRI, ultrasound, and positron emission tomography (PET) scans are used to rule out other neurodegenerative diseases. According to the present discovery, microglia in the brain may express high levels of BRI3. Therefore, incorporating BRI3 imaging during such imaging tests will allow for more accurate diagnosis of PD. Various ligand-specific brain image technologies are available (e.g., Sehlin D. et al., “Engineered antibodies: new possibilities for brain PET?”, Eur J Nucl Med Mol Imaging, 2019 December; 46 (13): 2848-2858).

In some examples, a subject will receive an antibody fragment-based BRI3-specific agent (e.g., radiolabeled anti-BRI3 F (ab′) 2 fragment conjugated to an anti-transferrin receptor (TfR) antibody) and then be subject to PET scan. If the patient brain is stained positive with the agent, the subject will be diagnosed with PD.

When diagnosing, regardless of whether the diagnosis is based on the transcriptome, protein expression, or exosomal analyses, or neuroimaging, the stage and/or disease progression may be further determined based on the degree of gene upregulation (e.g. of BRI3) and/or downregulation and/or the degree of changes in the immune population. Furthermore, these analyses are applicable not only to diagnosis of PD but also to determination of whether a subject has inflammation associated with a neurodegenerative disease such as PD.

Once diagnosed with PD and/or neural inflammation, the subject may be administered with one or more therapeutics for PD and/or neural inflammation. Non-limiting examples of the therapeutic include levodopa, carbidopa, a dopamine agonist (e.g., pramipexole, ropinirole, rotigotine, apomorphine), a monoamine oxidase B (MAO B) inhibitor (e.g., selegiline, rasagiline, safinamide), a catechol O-methyltrasnferase (COMT) inhibitor (e.g., entacapone, tolcapone), an anticholinergic (e.g., benztropine), amantadine, an analgesic, a corticosteroid, an anti-inflammatory, a microglial suppressor, a neuroregenerating agent (e.g., vitamin B12, chorionic gonadotropin), a neurohormone (e.g., oxytocin), or any combination thereof, further optionally comprising administering deep brain stimulation (DBS).

### Example 3: Detection of Disease Progression in Patients

Changes in the gene expression and/or immune cell populations are also useful in monitoring disease progression in a patient who already has a neurodegenerative disease (e.g., whether the severity and/or stage of PD has advanced, and/or inflammatory levels have increased).

Blood and/or CSF samples will be obtained from a subject contracted with a neurodegenerative disease such as PD at multiple timepoints (e.g., about once per three months, about once per 6 months, about once per year, etc). Transcript and/or protein expression of genes (e.g., BRI3) and/or immune cell population will be analyzed as described in Example 2.

If BRI3 expression as transcript and/or protein has significantly increased relative to a previous timepoint, for example in monocytes (e.g., CD16⁺ monocytes and/or CD14⁺ monocytes), it will be determined that the disease severity and/or stage has advanced and/or inflammatory levels have increased. In some cases, expression of other genes and immune cell population (proportion) will be also determined as in Example 2 and used towards determining the progression. Nucleic acid and/or protein contents in exosomes isolated from the blood and/or CSF samples may also be also analyzed and utilized toward determining disease progression.

### Example 4: BRI3 Inactivation Modifies Monocyte Phenotype

Four independently established control and BRI3 null THP-1 cultures were generated using CRISPR/CAS9. Cultures were differentiated using phorbol 12-myristate 13-acetate (PMA) and gene expression was evaluated using the nanostring platform. As shown in FIGS. 7A, and 7B, pathway scoring identified an elevation in fatty acid synthesis score and a reduction in arginine metabolism score in normalized data from BRI3 inactivated cultures compared with control. FIG. 7C shows that reduced expression of CD84 was seen in BRI3 inactivated cultures compared with control (p=0.0010, one way ANOVA with Sidak's multiple comparisons test.

As shown herein BRI3 is robustly elevated in CD16 monocytes in Parkinson's disease (PD) patients. CD16 expression indicates either an intermediate or non-classical “M2” monocyte state with tissue invasion, pro-inflammatory, and anti-inflammatory potential. Based on our results we believe that BRI3 modulates monocyte gene expression following PMA directed differentiation into the CD16 phenotype. This is supported by other identified significant alterations in genes required for phenotypic changes related to the M2 phenotype. These include arginase metabolism, fatty acid synthesis, and expression of the vascular adhesion molecule CD84 as these activities are associated with peripheral monocyte phenotypic conversion and trafficking into diseased tissues. Based thereon reducing BRI3 expression and/or activity may be useful in inhibiting the production of M2 monocytes and/or the migration/infiltration of such monocytes into inflamed sites such as in the CNS of subjects with or at risk of developing neuroinflammatory conditions.

What is claimed is:

1. A method of preventing or inhibiting the differentiation of monocytes
Into the M2 phenotype and/or for preventing or inhibiting the trafficking or migration of monocytes, e.g., M2 monocytes, into inflammatory sites in a subject in need thereof by inhibiting/preventing the expression and/or function of BRI3 in the subject.

2. The method of claim 1, wherein
(i) the subject has or is at risk of developing a chronic or acute inflammatory condition;
(ii) the subject has or is at risk of developing a chronic or acute neuroinflammatory condition;
(iii) the acute or chronic inflammatory condition is caused by an aging, infection, an environmental insult, an injury such as a repetitive motion injury or trauma injury, or an autoimmune or inflammatory condition;
(iv) the acute or chronic neuroinflammatory condition is caused by aging, an infection, an environmental insult, an injury such as a traumatic brain injury, an autoimmune condition, a sterile inflammatory condition, a demyelinating condition, stress, stroke or a neurodegenerative disease or other inflammatory condition that affects the central nervous system (CNS);
(v) the subject is at risk of developing the inflammatory condition because of heredity, environmental exposure or an activity such as a sports activity correlated to a neuroinflammatory condition;
(vi) the infection of (iv) is caused by a virus, bacteria, parasite, yeast or fungus;
(vii) the infection of (iv) is caused by a coronavirus (e.g., SARS-COVID or SARS COVID 2 (Covid 19)), Japanese encephalitis virus, influenza virus, measles virus, herpes simplex virus, varicella zoster virus, mumps virus, chicken pox virus, rubella virus, Streptococcus pneumoniae, Mycoplasma pneumoniae, human metapneumovirus (HPMV), human parainfluenza virus, Legionnaires' disease, respiratory syncytial virus (RSV), rhinovirus, pneumocystis pneumonia, or pneumococcal disease;
(viii) the neurodegenerative disease of (iv) is selected from Alzheimer's disease, Parkinson's disease (PD), senility or another memory disorder, ataxia, motor neuron disease, multiple sclerosis (MS), Lewy body disease or Lewy body dementia (LBD), multiple system atrophy (MSA), progressive supranuclear palsy (PSP), amyotrophic lateral sclerosis (ALS) or motor neurone diseases (MND), Huntington's Disease (HD), spinocerebellar ataxia (SCA), Friedreich's ataxia (FA), spinal muscular atrophy (SMA), or prion disease (e.g., Creutzfeldt-Jakob disease (CJD)), optionally wherein the neurodegenerative diseases is PD;
(ix) the demyelinating condition of (iv) is selected from multiple sclerosis, ALS, Transverse Myelitis, Guillain-Barre syndrome, Neuromyelitis optica, or Devic's disease, an Idiopathic inflammatory demyelinating disease, a Leukodystrophic or dysmyelinating disorder, Central pontine myelinolysis, a myelopathy such as tabes dorsalis (syphilitic myelopathy), Leukoencephalopathy such as progressive multifocal leukoencephalopathy, a Leukodystrophy, chronic inflammatory demyelinating polyneuropathy
Anti-MAG peripheral neuropathy, Charcot-Marie-Tooth disease, Hereditary neuropathy and Progressive inflammatory neuropathy;
(x) the autoimmune disease of (iii) that affects the CNS is selected from Neuromyelitis optica, Anti-myelin oligodendrocyte glycoprotein antibody disease (MOG), Acute disseminated encephalomyelitis (ADEM), Chronic meningitis, Central nervous system (CNS) vasculitis, Hashimoto's encephalitis, Steroid responsive encephalopathy associated with autoimmune thyroiditis (SREAT), Neurosarcoidosis, Optic neuritis, Transverse myelitis;
(xi) the chronic or acute inflammation is caused by a vaccine; or
(xii) any combination of the foregoing

3. The method of claim 1, wherein the chronic or acute inflammation is caused by concussive injuries such as caused by a sports activity or by stroke or sepsis or acute respiratory disease syndrome (ARDS).

4. The method of claim 1, which further includes administering an active agent that modifies the expression and/or function of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof.

5. The method of claim 1, wherein the expression and/or function of BRI3 or any of the genes recited in claim 4 is reduced by about 25%, about 30%, about 40%, about 45%, about 50%, about 55% about 60%, about 65%, about 70%, about 75%, about 80%, about 85%, about 90%, about 95%, or about 100%.

6. The method of claim 1, wherein an active agent is also administered which increases the expression and/or function of HDDC2, EVL, UBB, RAC2, OAS1, LSP1, or ARF5, or any combination thereof, optionally wherein the increase is by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

7. The method of claim 1, wherein BRI3 expression or activity is reduced in peripheral blood mononuclear cells (PBMCs), monocytes, dendritic cells, and/or central nervous system (CNS) cells, optionally wherein the monocytes are circulating monocytes, further optionally wherein the monocytes are CD16⁺ monocytes and/or CD14⁺ monocytes, and yet further optionally wherein the CNS cells comprise or are microglia.

8. The method of claim 1, wherein the active agent reduces the expression and/or function of BRI3 in monocytes, optionally wherein the monocytes are circulating monocytes, further optionally wherein the monocytes are CD16⁺ monocytes CD14⁺ monocytes, and/or meningeal monocytes.

9. The method of claim 1, wherein the active agent that modifies the expression and/or function of BRI3 or other gene comprises a clustered Regularly Interspaced Short Palindromic Repeat (CRISPR)/Cas gene editing agent, a zinc-finger nuclease (ZFN) gene editing agent, a transcription activator-like effector nuclease (TALEN) gene editing agent, a transposase-based gene therapy, an siRNA, an shRNA, an miRNA, an aptamer, an antibody, an antigen-binding antibody fragment (e.g., scFv, Fab, Fab′, (Fab′) 2), a chimeric antigen receptor (CAR)-expressing cell, a peptide, a small molecule, a polymer, an expression vector encoding a gene of interest, or any combination thereof

10. The method of claim 1, wherein the active agent:
(i) comprises a CRISPR/Cas gene editing agent against BRI3;
(ii) comprises or consists of a short-guide RNA (sgRNA) selected from the oligonucleotide sequences AACTCTATCGTGGTCGTAGG (SEQ ID NO:1), CGTCACAGGTGGGCCCGTAA (SEQ ID NO:2), GACTACGCGTGCGGCCCGCA (SEQ ID NO: 3) (depicted here in “sense” orientation),
(iii) comprises or consists of a sgRNA targeting BRI3 falling within or including any of the following genomic sequences: GAGGAAGCGACGATGCCCCAACTGTGGAGC (SEQ ID NO:4), ACCACGATAGAGTTGGCAGGATAGCGGGTG (SEQ ID NO:5), AGTTGGGGCATCGTCGCTTCCTCAAGGCAA (SEQ ID NO:6), TTACGGGCCCACCTGTGACGAGGTAGGGGT (SEQ ID NO:7), GCCCTACCCCTACCTCGTCACAGGTGGGCC (SEQ ID NO:8), TCCTGCCAACTCTATCGTGGTCGTAGGAGG (SEQ ID NO:9), CCCGCTATCCTGCCAACTCTATCGTGGTCG (SEQ ID NO:10), GGGCGACTACGCGTGCGGCCCGCACGGCTA (SEQ ID NO:11), GCCCACCTGTGACGAGGTAGGGGTAGGGCG (SEQ ID NO:12), CCCAGGGTCTACAACATCCACAGCCGGACC (SEQ ID NO:13), CCACGATAGAGTTGGCAGGATAGCGGGTGA (SEQ ID NO:14), TTGGCAGGATAGCGGGTGACGGTCCGGCTG (SEQ ID NO:15), CAGGGATACCCACCCACCATCCCAGGGTCT (SEQ ID NO:16), CCTGGTGTTCCCTTTAAGCGAAGGTGGCTC (SEQ ID NO:17) (as annotated in Gene ID 25798, NM_015379.5, or identical BRI3 sequences in prior or future annotations),
(iv) comprises a sgRNA sequence selected from one comprising or consisting of any of the following nucleic acid sequences:
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 4)
GAGGAAGCGACGATGCCCCAACTGTGGAGC
(SEQ ID NO: 5)
ACCACGATAGAGTTGGCAGGATAGCGGGTG
(SEQ ID NO: 6)
AGTTGGGGCATCGTCGCTTCCTCAAGGCAA
(SEQ ID NO: 7)
TTACGGGCCCACCTGTGACGAGGTAGGGGT
(SEQ ID NO: 8)
GCCCTACCCCTACCTCGTCACAGGTGGGCC
(SEQ ID NO: 9)
TCCTGCCAACTCTATCGTGGTCGTAGGAGG
(SEQ ID NO: 10)
CCCGCTATCCTGCCAACTCTATCGTGGTCG
(SEQ ID NO: 11)
GGGCGACTACGCGTGCGGCCCGCACGGCTA
(SEQ ID NO: 12)
GCCCACCTGTGACGAGGTAGGGGTAGGGCG
(SEQ ID NO: 13)
CCCAGGGTCTACAACATCCACAGCCGGACC
(SEQ ID NO: 14)
CCACGATAGAGTTGGCAGGATAGCGGGTGA
(SEQ ID NO: 15)
TTGGCAGGATAGCGGGTGACGGTCCGGCTG
(SEQ ID NO: 16)
CAGGGATACCCACCCACCATCCCAGGGTCT
(SEQ ID NO: 17)
CCTGGTGTTCCCTTTAAGCGAAGGTGGCTC
(SEQ ID NO: 18)
AGCGGCCGCCCGCCTACAACCTGGAGGCCG
(SEQ ID NO: 19)
ACAGGGATACCCACCCACCATCCCAGGGTC
(SEQ ID NO: 20)
TAAGCGAAGGTGGCTCCACAGTTGGGGCAT
(SEQ ID NO: 21)
CCAAAGGGGAAGAGGATGATGGCCAGGAAG
(SEQ ID NO: 22)
TGGGATGGTGGGTGGGTATCCCTGTGGGCG
(SEQ ID NO: 23)
CAGCAAATGAACCCAAAGGGGAAGAGGATG
(SEQ ID NO: 24)
TACGGGCCCACCTGTGACGAGGTAGGGGTA
(SEQ ID NO: 25)
CTACGCGTGCGGCCCGCACGGCTACGGCGC
(SEQ ID NO: 26)
GGCGGGCGGCCGCTCCTGCAGCAGCGGCTT
(SEQ ID NO: 27)
GACCACAAGCCGCTGCTGCAGGAGCGGCCG
(SEQ ID NO: 28)
CTGCCCGTCTCTGCTGCAGGGTTGGGGTGC
(SEQ ID NO: 29)
CCCACCTGTGACGAGGTAGGGGTAGGGCGG
(SEQ ID NO: 30)
TGATGGCCAGGAAGATGCCCAGGAAGGTGA
(SEQ ID NO: 31)
GGGCCTGGTGTTCCCTTTAAGCGAAGGTGG
(SEQ ID NO: 32)
CTGTGGATGTTGTAGACCCTGGGATGGTGG
(SEQ ID NO: 33)
AGGCAAAACAGCAAATGAACCCAAAGGGGA
(SEQ ID NO: 34)
GCCGCCCTACCCCTACCTCGTCACAGGTGG
(SEQ ID NO: 35)
GCAAAACAGCAAATGAACCCAAAGGGGAAG
(SEQ ID NO: 36)
GTCGTAGGAGGCTGTCCTGTCTGCAGGTGA
(SEQ ID NO: 37)
GGCCGGCCAGGGCGACTACGCGTGCGGCCC
(SEQ ID NO: 38)
GGCCGCTCCTGCAGCAGCGGCTTGTGGTCC
(SEQ ID NO: 39)
GGCAAAACAGCAAATGAACCCAAAGGGGAA
(SEQ ID NO: 40)
CGCCTACAACCTGGAGGCCGGCCAGGGCGA
(SEQ ID NO: 41)
TGCGGGCCGCACGCGTAGTCGCCCTGGCCG
(SEQ ID NO: 42)
CCTGCAGCAGCGGCTTGTGGTCCATGGCGG
(SEQ ID NO: 43)
TCTGCTGCAGGGTTGGGGTGCTGGAGGACT
(SEQ ID NO: 44)
GCCGGCCTCCAGGTTGTAGGCGGGCGGCCG
(SEQ ID NO: 45)
CCGGCTGTGGATGTTGTAGACCCTGGGATG
(SEQ ID NO: 46)
CCATGGACCACAAGCCGCTGCTGCAGGAGC
(SEQ ID NO: 47)
TGTGACGAGGTAGGGGTAGGGCGGCGGCGG
(SEQ ID NO: 48)
TGGATGTTGTAGACCCTGGGATGGTGGGTG
(SEQ ID NO: 49)
AGGAGCGGCCGCCCGCCTACAACCTGGAGG
(SEQ ID NO: 50)
CTGGCCGGCCTCCAGGTTGTAGGCGGGCGG
(SEQ ID NO: 51)
GGATGTTGTAGACCCTGGGATGGTGGGTGG
(SEQ ID NO: 52)
ACGAGGTAGGGGTAGGGCGGCGGCGGGGGC
(SEQ ID NO: 53)
AGGATGATGGCCAGGAAGATGCCCAGGAAG
(SEQ ID NO: 54)
CTCTGCCCGTCTCTGCTGCAGGGTTGGGGT
(SEQ ID NO: 55)
GACGAGGTAGGGGTAGGGCGGCGGCGGGGG
(SEQ ID NO: 56)
GTTGTAGACCCTGGGATGGTGGGTGGGTAT
(SEQ ID NO: 57)
GGCGGGGATGGCGCCGTAGCCGTGCGGGCC
(SEQ ID NO: 58)
GGGTTCATTTGCTGTTTTGCCTTGAGGAAG
(SEQ ID NO: 59)
CGAGGTAGGGGTAGGGCGGCGGCGGGGGCG
(SEQ ID NO: 60)
CCTGGCCGGCCTCCAGGTTGTAGGCGGGCG
(SEQ ID NO: 61)
CTGGCCATCATCCTCTTCCCCTTTGGGTTC
(SEQ ID NO: 62)
TGAACCCAAAGGGGAAGAGGATGATGGCCA
(SEQ ID NO: 63)
GAGGTAGGGGTAGGGCGGCGGCGGGGGCGC
(SEQ ID NO: 64)
GCCGCCCGCCTACAACCTGGAGGCCGGCCA
(SEQ ID NO: 65)
GGCCGCACGCGTAGTCGCCCTGGCCGGCCT
(SEQ ID NO: 66)
TGTTGTAGACCCTGGGATGGTGGGTGGGTA
(SEQ ID NO: 67)
GGATAGCGGGTGACGGTCCGGCTGTGGATG
(SEQ ID NO: 68)
AACTGTGGAGCCACCTTCGCTTAAAGGGAA
(SEQ ID NO: 69)
CCTGGCCATCATCCTCTTCCCCTTTGGGTT
(SEQ ID NO: 70)
TTAAGCGAAGGTGGCTCCACAGTTGGGGCA
(SEQ ID NO: 71)
TCTGCCCGTCTCTGCTGCAGGGTTGGGGTG
(SEQ ID NO: 72)
ACTGTGGAGCCACCTTCGCTTAAAGGGAAC
(SEQ ID NO: 73)
TTTAAGCGAAGGTGGCTCCACAGTTGGGGC
(SEQ ID NO: 74)
GCGGGGATGGCGCCGTAGCCGTGCGGGCCG
(SEQ ID NO: 75)
CGCCCTGGCCGGCCTCCAGGTTGTAGGCGG
(SEQ ID NO: 76)
TCCGGCTGTGGATGTTGTAGACCCTGGGAT
(SEQ ID NO: 77)
GCGTAGTCGCCCTGGCCGGCCTCCAGGTTG
(SEQ ID NO: 78)
CCGCCTACAACCTGGAGGCCGGCCAGGGCG
(SEQ ID NO: 79)
GCTGGAGGACTGCTTCACCTTCCTGGGCAT
(SEQ ID NO: 80)
GCTTCACCTTCCTGGGCATCTTCCTGGCCA
(SEQ ID NO: 81)
GCGGCGGCGGGGGCGCGGCGGGGATGGCGC
(SEQ ID NO: 82)
GTCTCTGCTGCAGGGTTGGGGTGCTGGAGG
(SEQ ID NO: 83)
TAGGGCGGCGGCGGGGGCGCGGCGGGGATG
(SEQ ID NO: 84)
TGCTGGAGGACTGCTTCACCTTCCTGGGCA
(SEQ ID NO: 85)
GGTAGGGCGGCGGCGGGGGCGCGGCGGGGA
(SEQ ID NO: 86)
AGGGGTAGGGCGGCGGCGGGGGCGCGGCGG
(SEQ ID NO: 87)
GTAGGGCGGCGGCGGGGGCGCGGCGGGGAT;
or
(v) comprises any anti-BRI3 agent utilizing CRISPR/Cas9 with an inactivated endonuclease, referred to as “dead” Cas9 or “dCas9.”

11. The method of claim 1, wherein the active agent comprises or consists of one or more short-guide RNAs having sequences selected from one or more of oligonucleotide sequences AACTCTATCGTGGTCGTAGG (SEQ ID NO:1), CGTCACAGGTGGGCCCGTAA (SEQ ID NO:2), GACTACGCGTGCGGCCCGCA (SEQ ID NO:3) (depicted here in “sense” orientation) and optionally inactivated endonuclease, further optionally “dead” Cas9 or “dCas9.”

12. The method of claim 1, further comprising administering at least one other active agent, optionally wherein the at least one other active agent is levodopa, carbidopa, a dopamine agonist (e.g., pramipexole, ropinirole, rotigotine, apomorphine), a monoamine oxidase B (MAO B) inhibitor (e.g., selegiline, rasagiline, safinamide), a catechol O-methyltrasnferase (COMT) inhibitor (e.g., entacapone, tolcapone), an anticholinergic (e.g., benztropine), or amantadine, or any combination thereof, further optionally comprising administering deep brain stimulation (DBS).

13. The method of claim 1, further comprising conducting a treatment which effects one or more of the following:
(i) increasing CD16⁺ monocytes;
(ii) increasing B cells;
(iii) increasing dendritic cells;
(iv) reducing CD14⁺ monocytes; and
(v) reducing CD4⁺ T cells,
optionally wherein the increasing and/or reducing in any one or more of (i)-(v) at least takes place in PBMCs.

14. The method of claim 1, further comprising detecting the expression and/or function of BRI3 in monocytes of the subject, and optionally one or more of FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, the expression of which is to be increased or decreased, wherein said detecting occurs prior, during and/or after said administrating, optionally wherein the detecting comprises detecting in one or more samples from the treated subject, optionally a blood sample and/or a brain sample.

15. The method of claim 1, which further comprises determining whether the severity and/or stage of and/or inflammation, optionally associated with a neurodegenerative disease has decreased or increased in a subject before, after or during treatment, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject at a first time point and a second time point, and optionally measuring the quantity (number and/or percentage) of CD16⁺ monocytes, B cells, dendritic cells, CD14⁺ monocytes, and CD4⁺ T cells in the subject or in the sample at the first time point and the second time point; and
(b) determining that the severity and/or stage and/or inflammation has decreased or increased
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is lower or higher at the second time point than the first time point; and/or
(ii) the expression of HDDC2, EVL, UBB, RAC2, OAS1, LSP1, or ARF5, or any combination thereof is higher or lower at the second time point than the first time point,
and optionally if:
(iii) the quantity of CD16⁺ monocytes, B cells, and/or dendritic cells is lower at the second time point than the first time point; and/or
(iv) the quantity of CD14⁺ monocytes and/or CD4⁺ T cells is higher at the second time point than the first time point,
optionally wherein the neurodegenerative disease is PD,
further optionally wherein the method comprises one or more of the following:
(I) in (a), at least the expression of BRI3 is measured;
(II) in (b), at least the expression of BRI3 is higher at the second time point than the first time point;
(III) in (b) (i), the expression is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(IV) in (b) (ii), the expression is lower at the second time point than the first time point by about 25%, about 30%, about 40%, about 45%, about 50%, about 55% about 60%, about 65%, about 70%, about 75%, about 80%, about 85%, about 90%, about 95%, or about 100%;
(V) in (b) (iii), the quantity is lower at the second time point than the first time point by about 25%, about 30%, about 40%, about 45%, about 50%, about 55% about 60%, about 65%, about 70%, about 75%, about 80%, about 85%, about 90%, about 95%, or about 100%; and/or
(VI) in (b) (iv), the quantity is higher at the second time point than the first time point by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%.

16. A method of determining whether the method of claim 1, is effective in a subject, comprising:
(a) measuring the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, TUFM, HDDC2, EVL, UBB, RAC2, OAS1, LSP1, or ARF5, or any combination thereof in the subject or in a sample from the subject before and at one or more time points after starting the therapy, and optionally measuring the quantity (number and/or percentage) of CD16⁺ monocytes, B cells, dendritic cells, CD14⁺ monocytes, and CD4⁺ T cells in the subject or in the sample before and at one or more time points after starting the therapy; and
(b) determining that the therapy is effective
if:
(i) the expression of BRI3, FAM89B, PCBP1, ATP5F1E (or ATP5E), SH2B2, LMO2, TAF10, MAP2K2, TLE4, GRK6, RNF187, TNNT1, PTP4A2, SIAH2, YBX3, LAMP1, RPL8, EID2, CAPG, SRM, TMSB10, FYB, HLA-E, GLRX5, SEC61G, TMEM9, DBP, XRRA1, BLOC1S4, or TUFM, or any combination thereof is lower at least one time point after starting the therapy compared to before starting the therapy; and/or
(ii) the expression of HDDC2, EVL, UBB, RAC2, OAS1, LSP1, or ARF5, or any combination thereof is higher at least one time point after starting the therapy compared to before starting the therapy,
and optionally if:
(iii) the quantity of CD16⁺ monocytes, B cells, and/or dendritic cells is lower at least one time point after starting the therapy compared to before starting the therapy; and/or
(iv) the quantity of CD14⁺ monocytes and/or CD4⁺ T cells is higher least one time point after starting the therapy compared to before starting the therapy,
optionally wherein the neurodegenerative disease is PD,
further optionally wherein the method comprises one or more of the following:
(I) in (a), at least the expression of BRI3 is measured;
(II) in (b), at least the expression of BRI3 is lower at least one time point after starting the therapy compared to before starting the therapy;
(III) in (b) (i), the expression is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%;
(IV) in (b) (ii), the expression is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%;
(V) in (b) (iii), the quantity is higher at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, about 100%, about 150%, about 200%, about 300%, about 400%, about 500%, about 600%, about 800%, about 900%, about 1000%, about 2000%, about 3000%, about 4000%, about 5000%, or about 10000%; and/or
(VI) in (b) (iv), the quantity is lower at least one time point after starting the therapy compared to before starting the therapy by about 25%, about 30%, about 40%, about 45%, about 50%, about 55% about 60%, about 65%, about 70%, about 75%, about 80%, about 85%, about 90%, about 95%, or about 100%.

17. The method of claim 1, wherein, BRI3 or other gene detection measuring is effected by one or more of RNA sequencing, RNA-sequencing at single cell resolution, DNA array, flow cytometry, histochemistry, protein detection (optionally by use of bead-based or solid phase protein detection methods, further optionally wherein protein detection is effected by the use of one or more of an immunosorbent assay, gel electrophoresis, SDS-PAGE (polyacrylamide gel electrophoresis), Liquid chromatography-mass spectrometry (LC-MS), HPLC, ELISA, immunoelectrophoresis, immunostaining, Western blot, protein colorimetric assay, flow cytometry, electron microscopy, an enzyme assay, immune fluorescence, spectrophotometry, and the like) or imaging, optionally wherein the sample comprises PBMCs, monocytes, dendritic cells, and/or central nervous system (CNS) cells, optionally wherein the monocytes are circulating monocytes, further optionally wherein the monocytes are CD16⁺ monocytes, CD14⁺ monocytes, and/or meningeal monocytes, and yet further optionally wherein the CNS cells comprise microglia.

18. A method according to claim 1, which further includes the administration of another anti-inflammatory agent such as a steroid, corticosteroid, an NSAID such as buprofen, Naproxen, diclofenac, celecoxib, mefenamic acid, etoricoxib, indomethacin or high dose aspirin, a biologic such as IL-6 or TNF antagonist antibody, Embrel, Humira, Actarit, Adelmidrol, Allicin, Amixetrine, Amlexanox, Azerizin, Baricitinib, BMS-345541, BMS-470539, Bufexamac, Cannabichromene, Cannabidiol, Cepharanthine, Cyclopentenone prostaglandin, Dagrocorat, Dapansutrile, Diacerein, EF-24, Enterococcus durans, Epiestriol, Fluasterone, Fosdagrocorat, Hinokinin, Ibuproxam, Icatibant, Ligstroside, Lisofylline, Mapracorat, Meconopsis horridula, Mesalazine, Minocycline, Modafinil, Mofezolac, Nangibotide, NR58-3.14.3, Oleocanthal, Oleopicrin, Oleuropein, Omega-3 fatty acid, Pregnenolone, Prostaglandin inhibitor, Pseudopterosin E, Safotibant, Selective glucocorticoid receptor modulator, Semapimod, Shea butter, a Statin, Tetramethylpyrazine, Toreforant, Trofinetide, Upadacitinib, Vinyldithiin, or VUF-600 or any combination thereof.


SEQUENCE LISTING (87 sequences)

SEQ ID NO: 1 - DNA, 20 bp, synthetic construct
  misc_feature 1..20: note = short-guide sgRNA
  source 1..20: mol_type = other DNA
aactctatcgtggtcgtagg

SEQ ID NO: 2 - DNA, 20 bp, synthetic construct
  misc_feature 1..20: note = short-guide sgRNA
  source 1..20: mol_type = other DNA
cgtcacaggtgggcccgtaa

SEQ ID NO: 3 - DNA, 20 bp, synthetic construct
  misc_feature 1..20: note = short-guide sgRNA
  source 1..20: mol_type = other DNA
gactacgcgtgcggcccgca

SEQ ID NO: 4 - DNA, 30 bp, synthetic construct
  misc_feature 1..30: note = sgRNA targeting BRI3
  source 1..30: mol_type = other DNA
gaggaagcgacgatgccccaactgtggagc

SEQ ID NO: 5 - DNA, 30 bp, synthetic construct
  misc_feature 1..30: note = sgRNA targeting BRI3
  source 1..30: mol_type = other DNA
accacgatagagttggcaggatagcgggtg

SEQ ID NO: 6 - DNA, 30 bp, synthetic construct
  misc_feature 1..30: note = sgRNA targeting BRI3
  source 1..30: mol_type = other DNA
agttggggcatcgtcgcttcctcaaggcaa

SEQ ID NO: 7 - DNA, 30 bp, synthetic construct
  misc_feature 1..30: note = sgRNA targeting BRI3
  source 1..30: mol_type = other DNA
ttacgggcccacctgtgacgaggtaggggt

SEQ ID NO: 8 - DNA, 30 bp, synthetic construct
  misc_feature 1..30: note = sgRNA targeting BRI3
  source 1..30: mol_type = other DNA
gccctacccctacctcgtcacaggtgggcc

SEQ ID NO: 9 - DNA, 30 bp, synthetic construct
  misc_feature 1..30: note = sgRNA targeting BRI3
  source 1..30: mol_type = other DNA
tcctgccaactctatcgtggtcgtaggagg

SEQ ID NO: 10 - DNA, 30 bp, synthetic construct
  misc_feature 1..30: note = sgRNA targeting BRI3
  source 1..30: mol_type = other DNA
cccgctatcctgccaactctatcgtggtcg

SEQ ID NO: 11 - DNA, 30 bp, synthetic construct
  misc_feature 1..30: note = sgRNA targeting BRI3
  source 1..30: mol_type = other DNA
gggcgactacgcgtgcggcccgcacggcta

SEQ ID NO: 12 - DNA, 30 bp, synthetic construct
  misc_feature 1..30: note = sgRNA targeting BRI3
  source 1..30: mol_type = other DNA
gcccacctgtgacgaggtaggggtagggcg

SEQ ID NO: 13 - DNA, 30 bp, synthetic construct
  misc_feature 1..30: note = sgRNA targeting BRI3
  source 1..30: mol_type = other DNA
cccagggtctacaacatccacagccggacc

SEQ ID NO: 14 - DNA, 30 bp, synthetic construct
  misc_feature 1..30: note = sgRNA targeting BRI3
  source 1..30: mol_type = other DNA
ccacgatagagttggcaggatagcgggtga

SEQ ID NO: 15 - DNA, 30 bp, synthetic construct
  misc_feature 1..30: note = sgRNA targeting BRI3
  source 1..30: mol_type = other DNA
ttggcaggatagcgggtgacggtccggctg

SEQ ID NO: 16 - DNA, 30 bp, synthetic construct
  misc_feature 1..30: note = sgRNA targeting BRI3
  source 1..30: mol_type = other DNA
cagggatacccacccaccatcccagggtct

SEQ ID NO: 17 - DNA, 30 bp, synthetic construct
  misc_feature 1..30: note = sgRNA targeting BRI3
  source 1..30: mol_type = other DNA
cctggtgttccctttaagcgaaggtggctc

SEQ ID NO: 18 - DNA, 30 bp, synthetic construct
  misc_feature 1..30: note = sgRNA
  source 1..30: mol_type = other DNA
agcggccgcccgcctacaacctggaggccg

SEQ ID NO: 19 - DNA, 30 bp, synthetic construct
  misc_feature 1..30: note = sgRNA
  source 1..30: mol_type = other DNA
acagggatacccacccaccatcccagggtc

SEQ ID NO: 20 - DNA, 30 bp, synthetic construct
  misc_feature 1..30: note = sgRNA
  source 1..30: mol_type = other DNA
taagcgaaggtggctccacagttggggcat

SEQ ID NO: 21 - DNA, 30 bp, synthetic construct
  misc_feature 1..30: note = sgRNA
  source 1..30: mol_type = other DNA
ccaaaggggaagaggatgatggccaggaag

SEQ ID NO: 22 - DNA, 30 bp, synthetic construct
  misc_feature 1..30: note = sgRNA
  source 1..30: mol_type = other DNA
tgggatggtgggtgggtatccctgtgggcg

SEQ ID NO: 23 - DNA, 30 bp, synthetic construct
  misc_feature 1..30: note = sgRNA
  source 1..30: mol_type = other DNA
cagcaaatgaacccaaaggggaagaggatg

SEQ ID NO: 24 - DNA, 30 bp, synthetic construct
  misc_feature 1..30: note = sgRNA
  source 1..30: mol_type = other DNA
tacgggcccacctgtgacgaggtaggggta

SEQ ID NO: 25 - DNA, 30 bp, synthetic construct
  misc_feature 1..30: note = sgRNA
  source 1..30: mol_type = other DNA
ctacgcgtgcggcccgcacggctacggcgc

SEQ ID NO: 26 - DNA, 30 bp, synthetic construct
  misc_feature 1..30: note = sgRNA
  source 1..30: mol_type = other DNA
ggcgggcggccgctcctgcagcagcggctt

SEQ ID NO: 27 - DNA, 30 bp, synthetic construct
  misc_feature 1..30: note = sgRNA
  source 1..30: mol_type = other DNA
gaccacaagccgctgctgcaggagcggccg

SEQ ID NO: 28 - DNA, 30 bp, synthetic construct
  misc_feature 1..30: note = sgRNA
  source 1..30: mol_type = other DNA
ctgcccgtctctgctgcagggttggggtgc

SEQ ID NO: 29 - DNA, 30 bp, synthetic construct
  misc_feature 1..30: note = sgRNA
  source 1..30: mol_type = other DNA
cccacctgtgacgaggtaggggtagggcgg

SEQ ID NO: 30 - DNA, 30 bp, synthetic construct
  misc_feature 1..30: note = sgRNA
  source 1..30: mol_type = other DNA
tgatggccaggaagatgcccaggaaggtga

SEQ ID NO: 31 - DNA, 30 bp, synthetic construct
  misc_feature 1..30: note = sgRNA
  source 1..30: mol_type = other DNA
gggcctggtgttccctttaagcgaaggtgg

SEQ ID NO: 32 - DNA, 30 bp, synthetic construct
  misc_feature 1..30: note = sgRNA
  source 1..30: mol_type = other DNA
ctgtggatgttgtagaccctgggatggtgg

SEQ ID NO: 33 - DNA, 30 bp, synthetic construct
  misc_feature 1..30: note = sgRNA
  source 1..30: mol_type = other DNA
aggcaaaacagcaaatgaacccaaagggga

SEQ ID NO: 34 - DNA, 30 bp, synthetic construct
  misc_feature 1..30: note = sgRNA
  source 1..30: mol_type = other DNA
gccgccctacccctacctcgtcacaggtgg

SEQ ID NO: 35 - DNA, 30 bp, synthetic construct
  misc_feature 1..30: note = sgRNA
  source 1..30: mol_type = other DNA
gcaaaacagcaaatgaacccaaaggggaag

SEQ ID NO: 36 - DNA, 30 bp, synthetic construct
  misc_feature 1..30: note = sgRNA
  source 1..30: mol_type = other DNA
gtcgtaggaggctgtcctgtctgcaggtga

SEQ ID NO: 37 - DNA, 30 bp, synthetic construct
  misc_feature 1..30: note = sgRNA
  source 1..30: mol_type = other DNA
ggccggccagggcgactacgcgtgcggccc

SEQ ID NO: 38 - DNA, 30 bp, synthetic construct
  misc_feature 1..30: note = sgRNA
  source 1..30: mol_type = other DNA
ggccgctcctgcagcagcggcttgtggtcc

SEQ ID NO: 39 - DNA, 30 bp, synthetic construct
  misc_feature 1..30: note = sgRNA
  source 1..30: mol_type = other DNA
ggcaaaacagcaaatgaacccaaaggggaa

SEQ ID NO: 40 - DNA, 30 bp, synthetic construct
  misc_feature 1..30: note = sgRNA
  source 1..30: mol_type = other DNA
cgcctacaacctggaggccggccagggcga

SEQ ID NO: 41 - DNA, 30 bp, synthetic construct
  misc_feature 1..30: note = sgRNA
  source 1..30: mol_type = other DNA
tgcgggccgcacgcgtagtcgccctggccg

SEQ ID NO: 42 - DNA, 30 bp, synthetic construct
  misc_feature 1..30: note = sgRNA
  source 1..30: mol_type = other DNA
cctgcagcagcggcttgtggtccatggcgg

SEQ ID NO: 43 - DNA, 30 bp, synthetic construct
  misc_feature 1..30: note = sgRNA
  source 1..30: mol_type = other DNA
tctgctgcagggttggggtgctggaggact

SEQ ID NO: 44 - DNA, 30 bp, synthetic construct
  misc_feature 1..30: note = sgRNA
  source 1..30: mol_type = other DNA
gccggcctccaggttgtaggcgggcggccg

SEQ ID NO: 45 - DNA, 30 bp, synthetic construct
  misc_feature 1..30: note = sgRNA
  source 1..30: mol_type = other DNA
ccggctgtggatgttgtagaccctgggatg

SEQ ID NO: 46 - DNA, 30 bp, synthetic construct
  misc_feature 1..30: note = sgRNA
  source 1..30: mol_type = other DNA
ccatggaccacaagccgctgctgcaggagc

SEQ ID NO: 47 - DNA, 30 bp, synthetic construct
  misc_feature 1..30: note = sgRNA
  source 1..30: mol_type = other DNA
tgtgacgaggtaggggtagggcggcggcgg

SEQ ID NO: 48 - DNA, 30 bp, synthetic construct
  misc_feature 1..30: note = sgRNA
  source 1..30: mol_type = other DNA
tggatgttgtagaccctgggatggtgggtg

SEQ ID NO: 49 - DNA, 30 bp, synthetic construct
  misc_feature 1..30: note = sgRNA
  source 1..30: mol_type = other DNA
aggagcggccgcccgcctacaacctggagg

SEQ ID NO: 50 - DNA, 30 bp, synthetic construct
  misc_feature 1..30: note = sgRNA
  source 1..30: mol_type = other DNA
ctggccggcctccaggttgtaggcgggcgg

SEQ ID NO: 51 - DNA, 30 bp, synthetic construct
  misc_feature 1..30: note = sgRNA
  source 1..30: mol_type = other DNA
ggatgttgtagaccctgggatggtgggtgg

SEQ ID NO: 52 - DNA, 30 bp, synthetic construct
  misc_feature 1..30: note = sgRNA
  source 1..30: mol_type = other DNA
acgaggtaggggtagggcggcggcgggggc

SEQ ID NO: 53 - DNA, 30 bp, synthetic construct
  misc_feature 1..30: note = sgRNA
  source 1..30: mol_type = other DNA
aggatgatggccaggaagatgcccaggaag

SEQ ID NO: 54 - DNA, 30 bp, synthetic construct
  misc_feature 1..30: note = sgRNA
  source 1..30: mol_type = other DNA
ctctgcccgtctctgctgcagggttggggt

SEQ ID NO: 55 - DNA, 30 bp, synthetic construct
  misc_feature 1..30: note = sgRNA
  source 1..30: mol_type = other DNA
gacgaggtaggggtagggcggcggcggggg

SEQ ID NO: 56 - DNA, 30 bp, synthetic construct
  misc_feature 1..30: note = sgRNA
  source 1..30: mol_type = other DNA
gttgtagaccctgggatggtgggtgggtat

SEQ ID NO: 57 - DNA, 30 bp, synthetic construct
  misc_feature 1..30: note = sgRNA
  source 1..30: mol_type = other DNA
ggcggggatggcgccgtagccgtgcgggcc

SEQ ID NO: 58 - DNA, 30 bp, synthetic construct
  misc_feature 1..30: note = sgRNA
  source 1..30: mol_type = other DNA
gggttcatttgctgttttgccttgaggaag

SEQ ID NO: 59 - DNA, 30 bp, synthetic construct
  misc_feature 1..30: note = sgRNA
  source 1..30: mol_type = other DNA
cgaggtaggggtagggcggcggcgggggcg

SEQ ID NO: 60 - DNA, 30 bp, synthetic construct
  misc_feature 1..30: note = sgRNA
  source 1..30: mol_type = other DNA
cctggccggcctccaggttgtaggcgggcg

SEQ ID NO: 61 - DNA, 30 bp, synthetic construct
  misc_feature 1..30: note = sgRNA
  source 1..30: mol_type = other DNA
ctggccatcatcctcttcccctttgggttc

SEQ ID NO: 62 - DNA, 30 bp, synthetic construct
  misc_feature 1..30: note = sgRNA
  source 1..30: mol_type = other DNA
tgaacccaaaggggaagaggatgatggcca

SEQ ID NO: 63 - DNA, 30 bp, synthetic construct
  misc_feature 1..30: note = sgRNA
  source 1..30: mol_type = other DNA
gaggtaggggtagggcggcggcgggggcgc

SEQ ID NO: 64 - DNA, 30 bp, synthetic construct
  misc_feature 1..30: note = sgRNA
  source 1..30: mol_type = other DNA
gccgcccgcctacaacctggaggccggcca

SEQ ID NO: 65 - DNA, 30 bp, synthetic construct
  misc_feature 1..30: note = sgRNA
  source 1..30: mol_type = other DNA
ggccgcacgcgtagtcgccctggccggcct

SEQ ID NO: 66 - DNA, 30 bp, synthetic construct
  misc_feature 1..30: note = sgRNA
  source 1..30: mol_type = other DNA
tgttgtagaccctgggatggtgggtgggta

SEQ ID NO: 67 - DNA, 30 bp, synthetic construct
  misc_feature 1..30: note = sgRNA
  source 1..30: mol_type = other DNA
ggatagcgggtgacggtccggctgtggatg

SEQ ID NO: 68 - DNA, 30 bp, synthetic construct
  misc_feature 1..30: note = sgRNA
  source 1..30: mol_type = other DNA
aactgtggagccaccttcgcttaaagggaa

SEQ ID NO: 69 - DNA, 30 bp, synthetic construct
  misc_feature 1..30: note = sgRNA
  source 1..30: mol_type = other DNA
cctggccatcatcctcttcccctttgggtt

SEQ ID NO: 70 - DNA, 30 bp, synthetic construct
  misc_feature 1..30: note = sgRNA
  source 1..30: mol_type = other DNA
ttaagcgaaggtggctccacagttggggca

SEQ ID NO: 71 - DNA, 30 bp, synthetic construct
  misc_feature 1..30: note = sgRNA
  source 1..30: mol_type = other DNA
tctgcccgtctctgctgcagggttggggtg

SEQ ID NO: 72 - DNA, 30 bp, synthetic construct
  misc_feature 1..30: note = sgRNA
  source 1..30: mol_type = other DNA
actgtggagccaccttcgcttaaagggaac

SEQ ID NO: 73 - DNA, 30 bp, synthetic construct
  misc_feature 1..30: note = sgRNA
  source 1..30: mol_type = other DNA
tttaagcgaaggtggctccacagttggggc

SEQ ID NO: 74 - DNA, 30 bp, synthetic construct
  misc_feature 1..30: note = sgRNA
  source 1..30: mol_type = other DNA
gcggggatggcgccgtagccgtgcgggccg

SEQ ID NO: 75 - DNA, 30 bp, synthetic construct
  misc_feature 1..30: note = sgRNA
  source 1..30: mol_type = other DNA
cgccctggccggcctccaggttgtaggcgg

SEQ ID NO: 76 - DNA, 30 bp, synthetic construct
  misc_feature 1..30: note = sgRNA
  source 1..30: mol_type = other DNA
tccggctgtggatgttgtagaccctgggat

SEQ ID NO: 77 - DNA, 30 bp, synthetic construct
  misc_feature 1..30: note = sgRNA
  source 1..30: mol_type = other DNA
gcgtagtcgccctggccggcctccaggttg

SEQ ID NO: 78 - DNA, 30 bp, synthetic construct
  misc_feature 1..30: note = sgRNA
  source 1..30: mol_type = other DNA
ccgcctacaacctggaggccggccagggcg

SEQ ID NO: 79 - DNA, 30 bp, synthetic construct
  misc_feature 1..30: note = sgRNA
  source 1..30: mol_type = other DNA
gctggaggactgcttcaccttcctgggcat

SEQ ID NO: 80 - DNA, 30 bp, synthetic construct
  misc_feature 1..30: note = sgRNA
  source 1..30: mol_type = other DNA
gcttcaccttcctgggcatcttcctggcca

SEQ ID NO: 81 - DNA, 30 bp, synthetic construct
  misc_feature 1..30: note = sgRNA
  source 1..30: mol_type = other DNA
gcggcggcgggggcgcggcggggatggcgc

SEQ ID NO: 82 - DNA, 30 bp, synthetic construct
  misc_feature 1..30: note = sgRNA
  source 1..30: mol_type = other DNA
gtctctgctgcagggttggggtgctggagg

SEQ ID NO: 83 - DNA, 30 bp, synthetic construct
  misc_feature 1..30: note = sgRNA
  source 1..30: mol_type = other DNA
tagggcggcggcgggggcgcggcggggatg

SEQ ID NO: 84 - DNA, 30 bp, synthetic construct
  misc_feature 1..30: note = sgRNA
  source 1..30: mol_type = other DNA
tgctggaggactgcttcaccttcctgggca

SEQ ID NO: 85 - DNA, 30 bp, synthetic construct
  misc_feature 1..30: note = sgRNA
  source 1..30: mol_type = other DNA
ggtagggcggcggcgggggcgcggcgggga

SEQ ID NO: 86 - DNA, 30 bp, synthetic construct
  misc_feature 1..30: note = sgRNA
  source 1..30: mol_type = other DNA
aggggtagggcggcggcgggggcgcggcgg

SEQ ID NO: 87 - DNA, 30 bp, synthetic construct
  misc_feature 1..30: note = sgRNA
  source 1..30: mol_type = other DNA
gtagggcggcggcgggggcgcggcggggat